(19) United States
(12) Patent Application Publication
(10) Pub. No.: US 2025/0308651 A1
(43) Pub. Date: Oct. 2, 2025

(54) EMS ELECTRONIC PATIENT CARE RECORD VALIDATION TOOL

(71) Applicant: ZOLL Medical Corporation, Chelmsford, MA (US)

(72) Inventors: Frederick W. Forester, Encinitas, CA (US); Alyssa T. Greene, Mars, PA (US); Aaron R. Hart, Arvada, CO (US); Michael E. Markidis, Santee, CA (US); Gordon P. Nall, San Diego, CA (US); Sri Sravya Rachakonda, San Diego, CA (US); Eric H. Strand, Marshfield, MA (US)

(73) Assignee: ZOLL Medical Corporation, Chelmsford, MA (US)

(21) Appl. No.: 19/016,116

(22) Filed: Jan. 10, 2025

Related U.S. Application Data
(60) Provisional application No. 63/571,045, filed on Mar. 28, 2024.

(51) Int. Cl. G16H 10/60; G16H 40/67

(52) U.S. Cl. CPC G16H 10/60; G16H 40/67

(57) ABSTRACT

A system for locally validating an electronic patient care record (ePCR). The system includes a server and a mobile computing device. The server is configured to convert a standard set of rules from the first syntax to a second syntax that is executable within an execution environment and to communicate the standard set of rules to the mobile computing device. The mobile computing device is configured to host, while operating in an offline mode, an ePCR application encoded in the second syntax. The mobile computing device is configured to execute, within the execution environment, at least a portion of the ePCR application to receive at least one ePCR data value via at least one user interface control, and to execute, within the execution environment, at least a portion of the standard set of rules encoded in the second syntax to validate the at least one ePCR data value.

### CROSS REFERENCE TO RELATED APPLICATIONS

This application claims priority under 35 U.S.C. § 119 (e) to U.S. Provisional Application Ser. No. 63/571,045, titled “EMS ELECTRONIC PATIENT CARE RECORD VALIDATION TOOL,” filed Mar. 28, 2024, which is hereby incorporated herein by reference in its entirety.

### BACKGROUND

The present disclosure is directed to systems and methods for emergency medical services (EMS) encounter recording. These systems and methods are crafted to provide efficient and accurate contemporaneous records within the constraints of an EMS environment.

EMS agencies create and use an electronic patient care record (ePCR) for each patient encounter. Even if a particular patient has been treated during multiple encounters with one or more EMS agencies, there will be a newly generated and separate ePCR for each encounter with each agency. This stands in contrast to a patient medical record generated by a physician where the record follows the patient and includes information about multiple encounters with the physician for that same patient. The ePCR contains a complete and time-stamped record of medical observations, interventions and treatments, and transport for the patient during a patient encounter. Due to the intricacies of medical care along with governmental reporting guidelines, the ePCR is typically a complex and lengthy document.

Software applications exist that interact with EMS personnel to complete ePCRs. These software applications include user interface screens with controls to receive input from EMS personnel regarding the patient encounter. This input specifies values of data fields that document the complete encounter record described above.

### SUMMARY

In at least one example, a system for locally validating an electronic patient care record (ePCR) is provided. The system can execute this local validation without connecting to a remote server during execution of the validation. The system includes a mobile computing device. The mobile computing device may include at least one first memory; at least one first network interface; at least one user interface device; and at least one first processor coupled with the at least one first memory, the at least one first network interface, and the at least one user interface device. The at least one first processor may be further configured to implement an ePCR application to cause the mobile computing device to provide an ePCR user interface, at the at least one user interface device, for capturing data values for the ePCR, the ePCR including a plurality of data fields, retrieve an electronic validation file from the at least one first memory, receive a plurality of data values via the ePCR user interface, each data value corresponding to a respective data field of the plurality of data fields, in response to receiving each data value, validate the data value at the mobile computing device, wherein to validate may include to apply at least a portion of a set of rules to an input to each data field as the data values are received and prior to completion of data entry and locking of the ePCR, generate a plurality of validation results based on validations of the data values, and provide indications of the validation results at the ePCR user interface. Validation results may include successful validations or unsuccessful validations (e.g., validation issues). Validation issues may include validation errors and/or validation warnings. In some examples, validation issues may be associated with simple explanations as to a reason for the error or warning. Alternatively or additionally, the validation issues may be associated with special reports that detail procedures that must be followed to comply with a particular validation rule.

In the system, the at least one first processor may be further configured to locally validate the data value at the mobile computing device. The at least one first processor may be further configured to locally validate the data value at the mobile computing device while the at least one first network interface is in a state of disconnection from a network configured to communicatively couple the mobile computing device to a remote server. The at least one first processor may be further configured to receive the plurality of data values via the ePCR user interface while the at least one first network interface is in the state of disconnection. The at least one first processor may be further configured to receive input instructing the at least one first processor to lock the ePCR; and lock the ePCR in response to reception of the input while the at least one first network interface is in the state of disconnection.

In the system, the electronic validation file may include a set of rules coded in a second syntax according to which the ePCR application is coded based on a conversion from a set of rules coded according to a first syntax. The first syntax may be incompatible with local validation. The first syntax may be a Schematron syntax and the second syntax may be a JAVASCRIPT syntax. The local validation may include a validation performed while the at least one first network interface is in a state of disconnection from a network configured to communicatively couple the mobile computing device to a remote server.

In the system, the validation results may include validation errors, and the indications of the validation results may include indications of the validation errors. The validation errors may include one or more of invalid data values or missing data values. The at least one first processor may be configured to validate a data value provided in response to the indications of validation errors prior to completion of data entry and locking of the ePCR. The mobile computing device may include a tablet or a smartphone. The user interface device may include a touchscreen.

In the system, the at least one first processor may be further configured to iteratively update the indications of validation results in response to receiving each data value. The indications of the validation results may include instructions for fixing a validation error. The indications of the validation results may include an error control configured to indicate a number of the validation results associated with a particular data set section of the ePCR, and in response to a user selection of the error control, indicate the validation results associated with the particular data set section of the ePCR. The at least one first processor may be further configured to provide the indications of the validation results at the ePCR user interface in proximity to controls of the ePCR user interface associated with the validation results. The at least one first processor may be further configured to provide the validation results at the ePCR user interface in a panel.

In the system, the indications of the validation results may include an access control configured to indicate a validation error associated with a data field of the plurality of data fields, and navigate, in response to a user selection of the access control, the ePCR user interface to a data set section associated with the data field. The access control may also be configured to indicate a validation error associated with a data field of the plurality of data fields; and receive, in response to a user selection of the access control, a new data value to store in the data field. The at least one first processor may be configured to provide the validation results at the ePCR user interface in an upload screen. In the system, the upload screen may include a plurality of type controls, each type control of the plurality of type controls being associated with a type of validation issue, and the at least one processor is configured to receive input selecting a type control of the plurality of type controls, and provide, at the ePCR user interface, at least one access control associated with the type of validation issue associated with the type control.

In the system, the set of rules may include standard validation rules. The standard validation rules may be applicable to determine whether an ePCR complies with a National Emergency Medical Services Information System (NEMSIS) standard. The set of rules may specify at least one message to be displayed if at least one validation result indicates at least one invalid value within at least one data field. The at least one message may include at least one custom message specified by a healthcare provider.

In the system, the at least one first processor may be further configured to store the validation results in the first memory; detect an inability to establish a connection to a remote server via the at least one first network interface; wait for a timeout period; establish, after the timeout period, a connection to the remote server via the at least one first network interface; and upload the validation results to the remote server. The plurality of data fields may include one or more of at least one trip information field, at least one patient demographics field, at least one medications administered field, at least one interventions performed field, or at least one medical history field. The at least one first processor may be further configured to determine the at least the portion of the set of rules to apply based on a data field and a position, within hierarchical information, of an authenticated identity of a user of the mobile computing device. The hierarchical information may include one or more of a country tier, a state tier, a region tier, a command tier, a facility tier, a service type tier, and a call type tier. The at least the portion of the set of rules may include a plurality of rules falling under a plurality of states specified in the state tier. The set of rules may include one or more logical implications.

In the system, to apply the portion of the set of rules may include to apply one rule to one value of one data field to generate one validation result. To apply the at least the portion of the set of rules may include to apply a plurality of rules to one or more values of one or more data fields to generate one or more validation results. To apply the plurality of rules to one or more values of one or more data fields to generate one or more validation results may include to iteratively apply individual rules of the plurality of rules to the one or more values of the one or more data fields to generate one or more validation results. To apply the at least the portion of the set of rules may include to implement code stored in the electronic validation file.

The system may further include a remote computing device. The remote computing device may include at least one second memory; at least one second network interface; and at least one second processor coupled with the at least one second memory and the at least one second network interface. The at least one second processor may be configured to retrieve, from the at least one second memory, a set of rules to validate ePCR data coded according to a first syntax; convert the set of rules from the first syntax to a second syntax according to which the ePCR application is coded; write the electronic validation file storing the set of rules coded in the second syntax; cause the at least one second network interface to communicatively couple with the at least one first network interface of the mobile computing device; and transmit the electronic validation file to the mobile computing device, wherein the mobile computing device may be further configured to store the electronic validation file in the at least one first memory.

In the system, to transmit the electronic validation file may include to receive a user input requesting transmission of the electronic validation file at the ePCR user interface provided at the mobile computing device, and transmit the electronic validation file in response to the user input requesting transmission. To transmit the electronic validation file may include to transmit the electronic validation file autonomously.

In the system, the set of rules may include a first set of rules; and the remote computing device may be further configured to implement a service including an administrative interface configured to receive a second set of rules, and merge the second set of rules with the first set of rules coded according to the second syntax prior to writing the electronic validation file. The second set of rules may be applicable to determine whether the ePCR complies with one or more policies of a healthcare provider. To merge the second set of rules with the first set of rules may include to identify at least one conflict between the first set of rules and the second set of rules; and communicate a message indicating the at least one conflict to an external process via the administrative interface. The administrative interface may include an administrative application. The administrative application may include a natural language user interface. The natural language user interface may include a chatbot.

In the system, to convert the set of rules may include to convert the set of rules from the first syntax to the second syntax. To convert may include to parse the set of rules coded according to the first syntax into an intermediate representation of the set of rules; and generate the set of rules coded according to the second syntax from the intermediate representation. The intermediate representation may include a syntax tree storing tokens extracted from the set of rules. The at least one second processor may be further configured to test at least a portion of the set of rules coded according to the second syntax; and to test may include to apply the portion to one or more values of one or more data fields of ePCR data to generate one or more validation results; and compare the one or more validation results to one or more expected results.

In another example, a method for validating an electronic patient care record (ePCR) is provided. The method includes installing, on a mobile computing device, an ePCR application configured to cause the mobile computing device to provide an ePCR user interface for capturing data values for an ePCR including a plurality of data fields; retrieving, by the mobile computing device, an electronic validation file from a memory of the mobile computing device; receiving a plurality of data values via the ePCR user interface while the mobile computing device is in a state of disconnection from a network configured to communicatively couple the mobile computing device with a remote computing device, each data value corresponding to a respective data field of the plurality of data fields; in response to receiving each data value, validating the data value while the mobile computing device in the state of disconnection, wherein validating may include applying at least a portion of a set of rules to the data value as the data value is received and prior to completing data entry and locking the ePCR; generating a plurality of validation results based on validations of the data values; and providing indications of the validation results at the ePCR user interface.

In the method, validating the data value may include locally validating the data value. The method may further include receiving input instructing the mobile computing device to lock the ePCR; and locking the ePCR in response to reception of the input while at least one network interface of the mobile computing device is in the state of disconnection. In the method, receiving the electronic validation file may include receiving a set of rules coded in a second syntax according to which the ePCR application is coded based on a conversion from a set of rules coded according to a first syntax, wherein the first syntax is incompatible with local validation. Receiving the set of rules may include receiving a set of rules coded in a JAVASCRIPT syntax based on a conversion of a set of rules coded in a Schematron syntax. Generating the plurality of validation results may include generating validation errors and providing the indication of the validation results may include providing indications of the validation errors. Generating the validation errors may include identifying one or more of invalid data values or missing data values.

The method may further include validating a data value provided in response to the indications of validation errors prior to completion of data entry and locking of the ePCR. In the method, installing the ePCR application may include installing the ePCR application on a tablet or a smartphone. Receiving the plurality of data values via the ePCR user interface may include receiving the plurality of data values via a touchscreen. Providing the indications of the validation results may include iteratively updating the indications of validation results in response to receiving each data value. Iteratively updating the indications may include providing instructions for fixing a validation error. Iteratively updating the indications may include indicating a number of the validation results associated with a particular data set section of the ePCR. Iteratively updating the indications may include providing the indications of the validation results at the ePCR user interface in proximity to controls of the ePCR user interface associated with the validation results. Iteratively updating the indications may include providing the validation results at the ePCR user interface in a panel.

In the method, providing the indications of the validation results may include indicating a validation error associated with a data field of the plurality of data fields; and navigating, in response to a user selection of the access control, the ePCR user interface to a data set section associated with the data field. Providing the indications of the validation results may include indicating a validation error associated with a data field of the plurality of data fields; and receiving, in response to a user selection of the access control, a new data value to store in the data field. Providing the indications of the validation results may include providing the validation results at the ePCR user interface in an upload screen. In the method, providing the validation results at the ePCR user interface in the upload screen may include providing a plurality of type controls, each type control of the plurality of type controls being associated with a type of validation issue; receiving input selecting a type control of the plurality of type controls; and providing, at the ePCR user interface, at least one access control associated with the type of validation issue associated with the type control.

In the method, applying the at least the portion of the set of rules may include applying standard validation rules. Applying the standard validation rules may include applying rules applicable to determine whether an ePCR complies with a National Emergency Medical Services Information System (NEMSIS) standard. Applying the at least the portion of the set of rules may include applying at least one rule that specifies at least one message to be displayed if at least one validation result indicates at least one invalid value within at least one data field. Applying the at least one rule may include applying at least one rule that specifies at least one custom message specified by a healthcare provider.

The method may further include storing the validation results in a memory of the mobile computing device; detecting an inability to establish a connection to a remote server via the network; waiting for a timeout period; establishing, after the timeout period, a connection to the remote server via the network; and uploading the validation results to the remote server. In the method, installing the ePCR application may include installing an ePCR application configured to cause the mobile computing device to provide an ePCR user interface for capturing data values for an ePCR including one or more of at least one trip information field, at least one patient demographics field, at least one medications administered field, at least one interventions performed field, or at least one medical history field.

The method may further include determining the at least the portion of the set of rules to apply based on a data field and a position, within hierarchical information, of an authenticated identity of a user of the mobile computing device. In the method, determining the at least the portion of the set of rules to apply may include determining the at least the portion of the set of rules to apply based on hierarchical information including one or more of a country tier, a state tier, a region tier, a command tier, a facility tier, a service type tier, and a call type tier. Determining the at least the portion of the set of rules to apply based on hierarchical information may include identifying a plurality of rules falling under a plurality of states specified in the state tier. Applying the at least the portion of the set of rules may include applying one or more logical implications. Applying the at least the portion of the set of rules may include applying one rule to one value of one data field to generate one validation result. Applying the at least the portion of the set of rules may include applying a plurality of rules to one or more values of one or more data fields to generate one or more validation results. Applying the plurality of rules to the one or more values of the one or more data fields to generate the one or more validation results may include iteratively applying individual rules of the plurality of rules to the one or more values of the one or more data fields to generate one or more validation results. Applying the at least the portion of the set of rules may include implementing code stored in the electronic validation file.

The method may further include retrieving, by a remote computing device, a set of rules to validate ePCR data coded according to a first syntax; converting, by the remote computing device, the set of rules from the first syntax to a second syntax according to which the ePCR application is coded; writing, by the remote computing device, the electronic validation file storing the set of rules coded in the second syntax; communicatively coupling, by the remote computing device, with the mobile computing device; transmitting, by the remote computing device, the electronic validation file to the mobile computing device; and storing, by the mobile computing device, the electronic validation file in the memory local to the mobile computing device. In the method, transmitting the electronic validation file may include receiving a user input requesting transmission of the electronic validation file at the ePCR user interface provided at the mobile computing device, and transmitting the electronic validation file in response to the user input requesting transmission. Transmitting the electronic validation file may include transmitting the electronic validation file autonomously.

In the method, retrieving the electronic validation file may include retrieving an electronic validation file storing a first set of rules; and the method further may include implementing a service including an administrative interface configured to receive a second set of rules, and merging the second set of rules with the first set of rules coded according to the second syntax prior to writing the electronic validation file. In the method, merging the second set of rules with the first set of rules may include merging a set of rules applicable to determine whether the ePCR complies with one or more policies of a healthcare provider with the first set of rules. Merging the second set of rules with the first set of rules may include identifying at least one conflict between the first set of rules and the second set of rules; and communicating a message indicating the at least one conflict to an external process via the administrative interface. Communicating the message via the administrative interface may include communicating a message via an administrative application. Communicating the message via the administrative application may include communicating a message via a natural language user interface. Communicating the message via the natural language user interface may include communicating a message via a chatbot.

In the method, converting the set of rules may include converting the set of rules from the first syntax to the second syntax. Converting may include to parsing the set of rules coded according to the first syntax into an intermediate representation of the set of rules; and generating the set of rules coded according to the second syntax from the intermediate representation. Parsing the set of rules into the intermediate representation may include parsing the set of rules into a syntax tree storing tokens extracted from the set of rules.

The method may further include testing at least a portion of the set of rules coded according to the second syntax, wherein testing may include applying the at least the portion to one or more data values of one or more data fields of ePCR data to generate one or more validation results, and comparing the one or more validation results to one or more expected results. Applying at least a portion of the set of rules may include executing the portion of the set of rules within a browser or a browser container.

In another example, a system for locally validating an electronic patient care record (ePCR) is provided. The system includes a server and a mobile computing device. The server is configured to receive a standard set of rules encoded in a first syntax, convert the standard set of rules from the first syntax to a second syntax that is executable within an execution environment, communicate the standard set of rules encoded in the second syntax to a mobile computing device configured to store an ePCR application encoded in the second syntax. The mobile computing device is configured to operate in an online mode, receive the standard set of rules encoded in the second syntax while operating in the online mode, shift to operate in an offline mode, execute, within the execution environment while operating in the offline mode, at least a portion of the ePCR application to receive at least one ePCR data value via at least one user interface control, execute, within the execution environment while operating in the offline mode, at least a portion of the standard set of rules encoded in the second syntax to validate the at least one ePCR data value prior to a focus of the ePCR application shifting away from the at least one user interface control, and display an indication of whether the at least one ePCR data value is valid prior to the focus of the ePCR application shifting away from the at least one user interface control. This in-line, as-you-go validation, or incremental validation contemporaneous with data entry, distinguishes some examples of the system from other systems that validate an entire ePCR or other medical record at or near the end of the record's creation and completion (e.g., just before locking an ePCR). The latter case is bulk validation, as opposed to incremental validation as described herein, and in sequence with and subsequent to data entry, as opposed to contemporaneous as described herein.

In the system, the first syntax may be a Schematron syntax, and the second syntax may be a JAVASCRIPT syntax. The execution environment may include a browser or a browser container. The indication may include a corrective prompt. The ePCR application may be further configured to validate and lock the ePCR regardless of a state of connection of the mobile computing device.

In the system, the mobile computing device may include at least one memory; at least one network interface; at least one user interface device; and at least one processor coupled with the at least one memory, the at least one network interface, and the at least one user interface device, the at least one processor configured to implement the ePCR application to cause the mobile computing device to provide, at the at least one user interface device, an ePCR user interface including the at least one user interface control, the ePCR user interface being configured to capture data values for the ePCR, the ePCR including a plurality of data fields, retrieve an electronic validation file from the at least one memory, receive a plurality of data values including the at least one ePCR data value via the ePCR user interface, each data value corresponding to a respective data field of the plurality of data fields, in response to receiving each data value, validate the data value at the mobile computing device, wherein to validate may include to apply at least a portion of a set of rules to an input to each data field as the data values are received and prior to completion of data entry and locking of the ePCR, generate a plurality of validation results based on validations of the data values, and provide indications of the validation results at the ePCR user interface, the indications including the indication of whether the at least one ePCR data value is valid.

In the system, the at least one processor may be further configured to locally validate the data value at the mobile computing device. The at least one processor may be further configured to locally validate the data value at the mobile computing device while the at least one network interface is in a state of disconnection from a network configured to communicatively couple the mobile computing device to a remote server. The at least one processor may be further configured to receive the plurality of data values via the ePCR user interface while the at least one network interface is in the state of disconnection. The at least one processor may be further configured to receive input instructing the at least one processor to lock the ePCR and lock the ePCR in response to reception of the input while the at least one network interface is in the state of disconnection. The electronic validation file may include a set of rules coded in a second syntax according to which the ePCR application is coded based on a conversion from a set of rules coded according to a first syntax, wherein the first syntax is incompatible with local validation. The first syntax may be a Schematron syntax and the second syntax may be a JAVASCRIPT syntax. The local validation may include a validation performed while the at least one network interface is in a state of disconnection from a network configured to communicatively couple the mobile computing device to a remote server.

In the system, the validation results may include validation errors, and the indications of the validation results may include indications of the validation errors. The validation errors may include one or more of invalid data values or missing data values.

In the system, the at least one processor may be further configured to validate a data value provided in response to the indications of validation errors prior to completion of data entry and locking of the ePCR. The mobile computing device may include a tablet or a smartphone. The user interface device may include a touchscreen. The at least one processor may be further configured to iteratively update the indications of validation results in response to receiving each data value. The indications of the validation results may include instructions for fixing a validation error. The indications of the validation results may include an error control configured to indicate a number of the validation results associated with a particular data set section of the ePCR, and in response to a user selection of the error control, indicate the validation results associated with the particular data set section of the ePCR. The at least one processor may be further configured to provide the indications of the validation results at the ePCR user interface in proximity to controls of the ePCR user interface associated with the validation results. The at least one processor may be further configured to provide the validation results at the ePCR user interface in a panel.

In the system, the indications of the validation results may include an access control configured to indicate a validation error associated with a data field of the plurality of data fields; and navigate, in response to a user selection of the access control, the ePCR user interface to a data set section associated with the data field. The access control may also be configured to indicate a validation error associated with a data field of the plurality of data fields; and receive, in response to a user selection of the access control, a new data value to store in the data field. The at least one first processor may be configured to provide the validation results at the ePCR user interface in an upload screen. In the system, the upload screen may include a plurality of type controls, each type control of the plurality of type controls being associated with a type of validation issue, and the at least one processor is configured to receive input selecting a type control of the plurality of type controls, and provide, at the ePCR user interface, at least one access control associated with the type of validation issue associated with the type control.

In the system, the set of rules may include standard validation rules. The standard validation rules may be applicable to determine whether an ePCR complies with a National Emergency Medical Services Information System (NEMSIS) standard. The set of rules may specify at least one message to be displayed if at least one validation result indicates at least one invalid value within at least one data field. The at least one message may include at least one custom message specified by a healthcare provider. The at least one processor may be further configured to store the validation results in the memory; detect an inability to establish a connection to a remote server via the at least one network interface; wait for a timeout period; establish, after the timeout period, a connection to the remote server via the at least one network interface; and upload the validation results to the remote server.

In the system, the plurality of data fields may include one or more of at least one trip information field, at least one patient demographics field, at least one medications administered field, at least one interventions performed field, or at least one medical history field. The at least one processor may be further configured to determine the at least the portion of the set of rules to apply based on a data field and a position, within hierarchical information, of an authenticated identity of a user of the mobile computing device. The hierarchical information may include one or more of a country tier, a state tier, a region tier, a command tier, a facility tier, a service type tier, and a call type tier. The at least the portion of the set of rules may include a plurality of rules falling under a plurality of states specified in the state tier. The set of rules may include one or more logical implications.

In the system, to apply the portion of the set of rules may include to apply one rule to one value of one data field to generate one validation result. To apply the at least the portion of the set of rules may include to apply a plurality of rules to one or more values of one or more data fields to generate one or more validation results. To apply the plurality of rules to one or more values of one or more data fields to generate one or more validation results may include to iteratively apply individual rules of the plurality of rules to the one or more values of the one or more data fields to generate one or more validation results. To apply the at least the portion of the set of rules may include to implement code stored in the electronic validation file.

In another example, one or more non-transitory, processor-readable storage media are provided. The media have stored thereon first processor-readable instructions for locally validating an electronic patient care record (ePCR). The first processor-readable instructions are configured to cause at least one first processor of a mobile computing device to implement an ePCR application to cause the mobile computing device to provide, at a user interface device, an ePCR user interface for capturing data values for the ePCR, the ePCR including a plurality of data fields; retrieve an electronic validation file from at least one first memory; receive a plurality of data values via the ePCR user interface, each data value corresponding to a respective data field of the plurality of data fields; in response to receiving each data value, validate the data value at the mobile computing device, wherein to validate may include to apply at least a portion of a set of rules to an input to each data field as the data values are received and prior to completion of data entry and locking of the ePCR; generate a plurality of validation results based on validations of the data values; and provide indications of the validation results at the ePCR user interface.

In the media, the first processor-readable instructions may be configured to cause the at least one first processor to locally validate the data value at the mobile computing device. The first processor-readable instructions may be configured to cause the at least one first processor to locally validate the data value at the mobile computing device while at least one first network interface is in a state of disconnection from a network configured to communicatively couple the mobile computing device to a remote server. The first processor-readable instructions may be configured to cause the at least one first processor to receive the plurality of data values via the ePCR user interface while the at least one first network interface is in the state of disconnection. The first processor-readable instructions may be configured to cause the at least one first processor to receive input instructing the at least one first processor to lock the ePCR and lock the ePCR in response to reception of the input while at the least one first network interface is in the state of disconnection.

In the media, the electronic validation file may include a set of rules coded according to a second syntax based on a conversion from a set of rules coded according to a first syntax, wherein the first syntax is incompatible with local validation and the first processor-readable instructions are coded in the second syntax. The first syntax may be a Schematron syntax and the second syntax may be a JAVASCRIPT syntax. The local validation may include a validation performed while at least one first network interface is in a state of disconnection from a network configured to communicatively couple the mobile computing device to a remote server. The validation results may include validation errors, and the indications of the validation results may include indications of the validation errors. The validation errors may include one or more of invalid data values or missing data values.

In the media, the first processor-readable instructions may be configured to cause the at least one first processor to validate a data value provided in response to the indications of validation errors prior to completion of data entry and locking of the ePCR. The mobile computing device may include a tablet or a smartphone. The user interface device may include a touchscreen. The first processor-readable instructions may be configured to cause the at least one first processor to iteratively update the indications of validation results in response to receiving each data value. The indications of the validation results may include instructions for fixing a validation error. The indications of the validation results may include an error control and the first processor-readable instructions may be configured to cause the error control to indicate a number of the validation results associated with a particular data set section of the ePCR, and in response to a user selection of the error control, indicate the validation results associated with the particular data set section of the ePCR. The indications of the validation results may include an access control configured to indicate a validation error associated with a data field of the plurality of data fields; and navigate, in response to a user selection of the access control, the ePCR user interface to a data set section associated with the data field. The indications of the validation results may include an access control configured to indicate a validation error associated with a data field of the plurality of data fields; and receive, in response to a user selection of the access control, a new data value to store in the data field. The first processor-readable instructions may be configured to cause the at least one first processor to provide the indications of the validation results at the ePCR user interface in proximity to controls of the ePCR user interface associated with the validation results. The first processor-readable instructions may be configured to cause the at least one first processor to provide the validation results at the ePCR user interface in a panel. The first processor-readable instructions may be configured to cause the at least one first processor to provide the validation results at the ePCR user interface in an upload screen. The upload screen may include a plurality of type controls, each type control of the plurality of type controls being associated with a type of validation issue; and the first processor-readable instructions may be configured to cause the at least one first processor to receive input selecting a type control of the plurality of type controls, and provide, at the ePCR user interface, at least one access control associated with the type of validation issue associated with the type control.

In the media, the set of rules may include standard validation rules. The standard validation rules may be applicable to determine whether an ePCR complies with a National Emergency Medical Services Information System (NEMSIS) standard. The set of rules may specify at least one message to be displayed if at least one validation result indicates at least one invalid value within at least one data field. The at least one message may include at least one custom message specified by a healthcare provider.

In the media, the first processor-readable instructions may be configured to cause the at least one first processor to store the validation results in the first memory; detect an inability to establish a connection to a remote server via at least one first network interface; wait for a timeout period; establish, after the timeout period, a connection to the remote server via the at least one first network interface; and upload the validation results to the remote server. The plurality of data fields may include one or more of at least one trip information field, at least one patient demographics field, at least one medications administered field, at least one interventions performed field, or at least one medical history field.

In the media, the first processor-readable instructions may be configured to cause the at least one first processor to determine the at least the portion of the set of rules to apply based on a data field and a position, within hierarchical information, of an authenticated identity of a user of the mobile computing device. The hierarchical information may include one or more of a country tier, a state tier, a region tier, a command tier, a facility tier, a service type tier, and a call type tier. The at least the portion of the set of rules may include a plurality of rules falling under a plurality of states specified in the state tier. The set of rules may include one or more logical implications.

In the media, the first processor-readable instructions to apply the portion of the set of rules may include first processor-readable instructions to apply one rule to one value of one data field to generate one validation result. The first processor-readable instructions to apply the at least the portion of the set of rules may include first processor-readable instructions to apply a plurality of rules to one or more values of one or more data fields to generate one or more validation results. The first processor-readable instructions to apply the plurality of rules to one or more values of one or more data fields to generate one or more validation results may include first processor-readable instructions to iteratively apply individual rules of the plurality of rules to the one or more values of the one or more data fields to generate one or more validation results. The first processor-readable instructions to apply the at least the portion of the set of rules may include first processor-readable instructions to implement code stored in the electronic validation file.

The media may further store second processor-readable instructions configured to cause at least one second processor of a remote server to retrieve a set of rules to validate ePCR data coded according to a first syntax; convert the set of rules from the first syntax to a second syntax according to which the first processor-readable instructions are coded; write the electronic validation file storing the set of rules coded in the second syntax; cause at least one second network interface to communicatively couple with at least one first network interface of the mobile computing device; and transmit the electronic validation file to the mobile computing device, wherein the mobile computing device may be further configured to store the electronic validation file in the at least one first memory.

In the media, the second processor-readable instructions to transmit the electronic validation file may include second processor-readable instructions to receive a user input requesting transmission of the electronic validation file at the ePCR user interface provided at the mobile computing device, and transmit the electronic validation file in response to the user input requesting transmission. The second processor-readable instructions to transmit the electronic validation file may include second processor-readable instructions to transmit the electronic validation file autonomously.

In the media, the set of rules may be a first set of rules; and the second processor-readable instructions may include second processor-readable instructions configured to cause the at least one second processor to implement a service including an administrative interface configured to receive a second set of rules, and merge the second set of rules with the first set of rules coded according to the second syntax prior to writing the electronic validation file. The second set of rules may be applicable to determine whether the ePCR complies with one or more policies of a healthcare provider. The second processor-readable instructions to merge the second set of rules with the first set of rules may include second processor-readable instructions to identify at least one conflict between the first set of rules and the second set of rules; and communicate a message indicating the at least one conflict to an external process via the administrative interface. The administrative interface may include an administrative application. The administrative application may include a natural language user interface. The natural language user interface may include a chatbot.

In the media, the second processor-readable instructions to convert the set of rules may include second processor-readable instructions to convert the set of rules from the first syntax to the second syntax. The second processor-readable instructions to convert may include the second processor-readable instructions to parse the set of rules coded according to the first syntax into an intermediate representation of the set of rules; and generate the set of rules coded according to the second syntax from the intermediate representation. The intermediate representation may include a syntax tree storing tokens extracted from the set of rules. The second processor-readable instructions may include second processor-readable instructions configured to cause the at least one second processor to test at least a portion of the set of rules coded according to the second syntax and the second processor-readable instructions to test may include second processor-readable instructions to apply the portion to one or more values of one or more data fields of ePCR data to generate one or more validation results; and compare the one or more validation results to one or more expected results.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples disclosed herein and are incorporated in and constitute a part of this specification. However, the figures are not intended to limit the scope of the disclosure. The figures, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1A illustrates front views of a login screen, a shift startup screen, and a chart selection screen provided by a mobile EMS charting application in accordance with at least one example disclosed herein.

FIG. 1B illustrates a front view of the new chart screen provided by a mobile EMS charting application in accordance with at least one example disclosed herein.

FIG. 2A is a block diagram illustrating an EMS charting system in accordance with at least one example disclosed herein.

FIG. 2B is a block diagram illustrating the EMS charting system of FIG. 2A in accordance with at least one example disclosed herein.

FIG. 3A is a block context diagram illustrating a validation service in accordance with at least one example disclosed herein.

FIG. 3B is a block diagram illustrating data structures implemented in memory by a validation service in accordance with at least one example disclosed herein.

FIG. 3C is a block diagram illustrating a conversion engine in accordance with at least one example disclosed herein.

FIG. 3D is a block diagram illustrating a transpiling process pipeline in accordance with at least one example disclosed herein.

FIG. 4A is a sequence diagram illustrating a process of generating and distributing rule sets in accordance with at least one example disclosed herein.

FIG. 4B is a sequence diagram illustrating another process of generating and distributing rule sets in accordance with at least one example disclosed herein.

FIG. 5 is a flow diagram illustrating a process of generating, distributing, and executing validation rule sets in accordance with at least one example disclosed herein.

FIG. 6A is a front view of a rules distribution screen in accordance with at least one example disclosed herein.

FIG. 6B is a front view of another rules distribution screen in accordance with at least one example disclosed herein.

FIG. 7A is a front view of an ePCR initial assessment screen with selected features rendered by validation code generated by a validation service in accordance with at least one example disclosed herein.

FIG. 7B is a front view of the ePCR initial assessment screen of FIG. 7A with selected features rendered by validation code generated by a validation service in accordance with at least one example disclosed herein.

FIG. 8A is a front view of another ePCR initial assessment screen with selected features rendered by validation code generated by a validation service in accordance with at least one example disclosed herein.

FIG. 8B is a front view of another ePCR initial assessment screen with selected features rendered by validation code generated by a validation service in accordance with at least one example disclosed herein.

FIG. 9 is a front view of a signatures screen in accordance with at least one example disclosed herein.

FIG. 10A is a front view of a share screen in accordance with at least one example disclosed herein.

FIG. 10B is a front view of an upload screen in accordance with at least one example disclosed herein.

FIG. 10C is a front view of another upload screen in accordance with at least one example disclosed herein.

FIG. 11 is a flow diagram of illustrating a process of creating and uploading an ePCR in accordance with at least one example disclosed herein.

FIG. 12 is a schematic diagram illustrating a mobile computing device in accordance with at least one example disclosed herein.

FIG. 13 is a schematic block diagram of examples of computing and medical device components with which at least one example disclosed herein may be implemented.

### DETAILED DESCRIPTION

As summarized above, the systems and methods disclosed herein validate ePCR data values efficiently and reliably in an EMS environment. Often in an emergency encounter, EMS caregivers interact with a critically ill patient for the first time and with no prior medical knowledge about the patient. The emergency encounter is usually in a non-medical environment like a home, office, or gym. In many cases, the encounter occurs in the chaotic environment of a fire scene, a car accident, or a mass casualty scene.

For example, consider an illustrative scenario of a crew of EMS caregivers in an ambulance being called upon to treat a patient suffering from an emergency medical condition (e.g., cardiac arrest, trauma, respiratory distress, drug overdose, etc.) and to transport the patient to a hospital. During the course of this emergency encounter, the EMS caregivers may be required to travel to a patient's scene, determine patient information, such as a mechanism of injury and a chief complaint, observe patient symptoms and conditions, measure patient physiological parameters (such as heart rate and other vital signs, electrocardiogram (ECG) traces, temperature, blood-oxygen data, and the like), administer treatments, interventions, and/or medications, and transport the patient from the scene to a medical facility.

To provide a complete and accurate record of each encounter that includes patient and encounter information, as described above, the EMS caregivers are tasked not only with providing medical care to patients but also recording and documenting detailed encounter information. For example, the EMS caregivers may document observations, examinations, and/or communications with the patient relevant to the patient's medical condition. This patient information can include, for instance, patient biographical information, past medical conditions, medications, allergies, vital signs, mental state, and the like. Other patient information recorded may include patient demographic information and billing/insurance information. In addition to patient information, the EMS caregivers may also be expected to record information regarding the encounter itself, such as the type of service requested, response mode, transport, and the like. This documentation may take the form of an ePCR. The ePCR includes data fields configured to store a comprehensive set of patient and encounter information according to a schema that controls the structure of the data provided to the digital record. The ePCR may include hundreds of mandatory data fields, although data field entries can include “not applicable” for data fields that do not apply to a particular call. In some examples, the schema may be a multi-agency standard that provides a compliance architecture to allow transfer of data and data interoperability between individual agency systems and enables entry of data in a centralized database. An example of such a standard is the National Emergency Medical Services Information Standard (NEMSIS) for emergency care medical record data collection. NEMSIS is an official EMS data collection standard for EMS agencies which allows transfer of data between systems and provides a national EMS repository for reporting and research. NEMSIS provides consistent definitions of data elements used in EMS and other pre-hospital care settings. The NEMSIS data collection via NEMSIS-compliant ePCRs may enable analysis of this data for evaluation of and evidence-based improvements in patient care across an array of EMS agencies. In particular, the NEMSIS-compliant ePCRs conform to a structured XML standard for the ePCR data. NEMSIS and the XML standard are examples only and other formats and/or content requirements are within the scope of this disclosure. For instance, the HL7®FHIR® (Health Level Seven Fast Healthcare Interoperability Resources) standard defines how healthcare information can be exchanged between different computer systems such as those servicing emergency care and those servicing hospitals. Other examples of standards include, but are not limited to, an HL7 version 2, version 3 or CDA standard, an Electronic Data Interchange (EDI) Healthcare including, 270, 271, 276, 277, 278, 820, 834, 835, 837P and 837I standard, SNOMED CT standard, diagnosis classification ICD standard, and procedure chart HCPCS and CPT standards.

In an implementation, the data fields within an ePCR may be organized into data set sections that cover various aspects of the emergency encounter. These data set sections may include, for example, data sets for airway, cardiac arrest, EMS crew, medical device, dispatch, patient disposition, patient examination, patient history, injury, laboratory results, and medications. In addition, in some implementations, an agency or other healthcare provider may include customized data set sections. As an example, a patient history section may include the data fields indicated below in Table 1. Examples of field values for the data fields are also provided in Table 1. The data field values may be associated with an ICD chart (e.g., International Classification of Diseases) for billing purposes.

TABLE 1

| Data field | Field value |
|---|---|
| Last name of Patient's Practitioner | Smith |
| First name of Patient's Practitioner | Chris |
| Advance Directives | none |
| Medication Allergies | Penicillin |
| Environmental/Food Allergies | Peanut |
| Medical/Surgical History | Type 2 diabetes |
| Medical History Obtained From | Patient's husband |
| Patient's Immunization | Flu |
| Immunization Year | Current year |
| Current Medications | Metformin |
| Current Medication Dose | 500 |
| Current Medication Dosage Unit | mg |
| Current Medication Administration | Oral |
| Alcohol/Drug Use | none |
| Pregnancy | yes |
| Last Oral Intake | none |

As another example of ePCR data, Table 2 below shows examples of data fields and data field values for a pre-scheduled dialysis transport.

TABLE 2

| Data field | Field value |
|---|---|
| Call Source | Phone call |
| Dispatch Center | Verifast EMS Services |
| Run Number | 47 |
| Incident Number | 56-87 |
| Dispatched Complaint | Palliative Care |
| Patient Acuity at Dispatch | Priority 4 (Non-Acute) |
| Changed Priority | Pre-Scheduled |
| Trauma call type | Medical and trauma |
| Call type | BLS |
| Response Mode | Pre-Scheduled |
| Additional Response Mode | No lights or Sirens |
| Pickup Zone | 16 |
| Response Delay | None |
| Type of Service | Interfacility transport |
| Patient Disposition | Treated & transported |

For maximum accuracy and to provide the most real-time benefit to caregivers, both pre-hospital and at a transport destination, the ePCR should be completed contemporaneously with, i.e., during, the ongoing encounter, with a completed and valid document available upon transfer of the patient from EMS to a medical facility. However, this documentation competes for the attention of the responders while the responders need to attend to medical care of the patient. In some implementations, the ePCR may include 50-1000 fields for which a data entry is required (e.g., required by laws of a state or another jurisdiction and/or required for adherence to a data collection standard).

ePCRs are subject to validation requirements that originate from a variety of sources, including national, state, local governments and EMS agencies. With this large number of sources and corresponding validation rules, EMS providers have come to rely on automated validation processes to help ensure that data values stored in ePCRs are valid and in compliance with applicable standards. However, the voluminous set of rules applicable to EMS calls creates a processing challenge for validation automation on small mobile computing devices preferred by EMS providers. Moreover, in many EMS care situations, the mobile computing device may lack network connectivity or be vulnerable to sporadic connectivity. For example, EMS care provided in a rural area, in a parking garage, in an urban canyon, or during transport may be subject to unavailable or unreliable network connectivity.

Thus, and in accordance with at least some examples disclosed herein, an EMS charting system is provided with ePCR data value validation that is optimized for the EMS environment. For instance, some examples disclosed herein distill the numerous validation rules described above, which may be encoded in differing syntaxes, into a single harmonized set of rules that is easier for mobile computing devices to apply and that allows for a simplified rule application architecture. The harmonized set of rules may include rules originating from NEMSIS and other national standards, as well as standards that are applicable to states and regions. The harmonized set of rules may further include customized validation rules authored by EMS agencies and other healthcare facilities. Moreover, in some examples, the harmonized set of rules are encoded in a particular syntax, such as JAVASCRIPT, that can be efficiently executed by the mobile computing devices, rather than requiring translation or other additional processing prior to execution. These technological improvements enable charting applications to validate ePCR data values as they are entered in real-time, thus increasing the speed with which EMS caregivers can complete and “lock” ePCRs. Locking an ePCR may include a variety of operations generally directed to ensuring the ePCR is complete, valid, and ready for subsequent use and review by healthcare providers or services other than the EMS providers responsible for originating the ePCR. For instance, in some examples, locking an ePCR may include validating (e.g., revalidating in whole or in part) that the ePCR complies with some or all applicable rule sets (e.g., a national rule set, one or more state rule sets, an agency rule set, etc.). For example, the system and methods described herein may provide real-time validation of ePCR entries as these entries are received and prior to locking the ePCR. However, the process of locking the ePCR may re-apply validation rules to the entire chart once a user of the ePCR has represented that the ePCR is complete as a final confirmation of the contents of the ePCR. The locking may further include recording that the ePCR is in a locked state (e.g., within the ePCR and/or elsewhere within the EMS charting system), and recording a timestamp indicative of the time the ePCR was locked. Locking an ePCR permits the EMS charting system to upload, immediately or at a later time, the ePCR for additional processing, such as autonomous and/or manual quality analysis, attachment of addendums to the ePCR, and modification of the ePCR by personnel other than the EMS caregivers. The process efficiencies introduced by the examples disclosed herein also enable ePCRs to be locked with confidence, regardless of whether the mobile computing device is connected to a network because the validation processes are comprehensive and executed locally by the mobile computing device. Moreover, local and efficient execution of the validation processes affords the systems and methods disclosed herein with the ability to provide corrective prompting to EMS caregivers to enable real-time corrections of validation errors and/or other validation issues, such as warnings. This corrective prompting may include simple reasons explaining why the validation issue exists and/or detailed instructions directed to the validation issue. The detailed instructions may be within a special report. Other advantages are discussed herein and will be apparent in view of this disclosure.

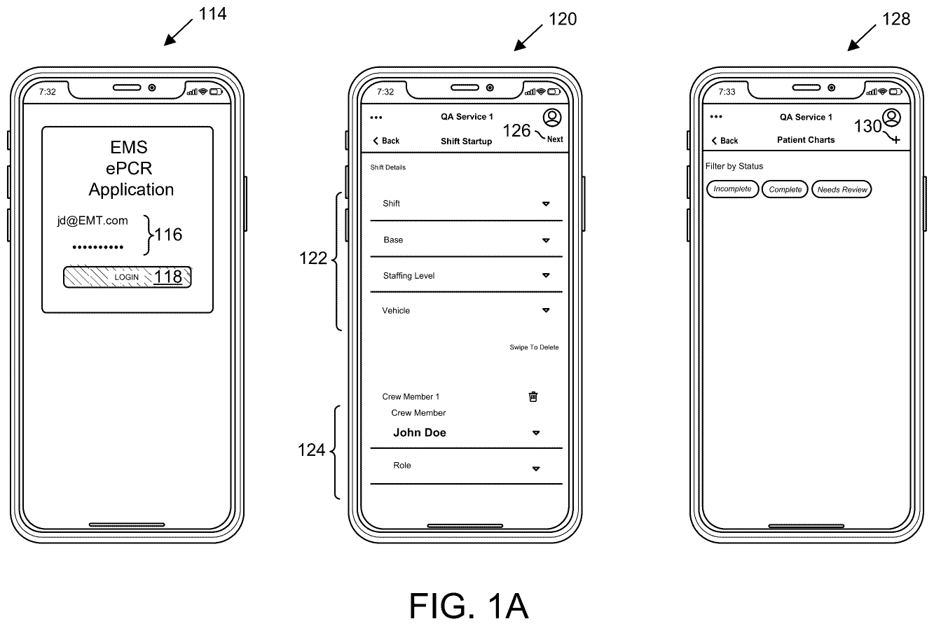

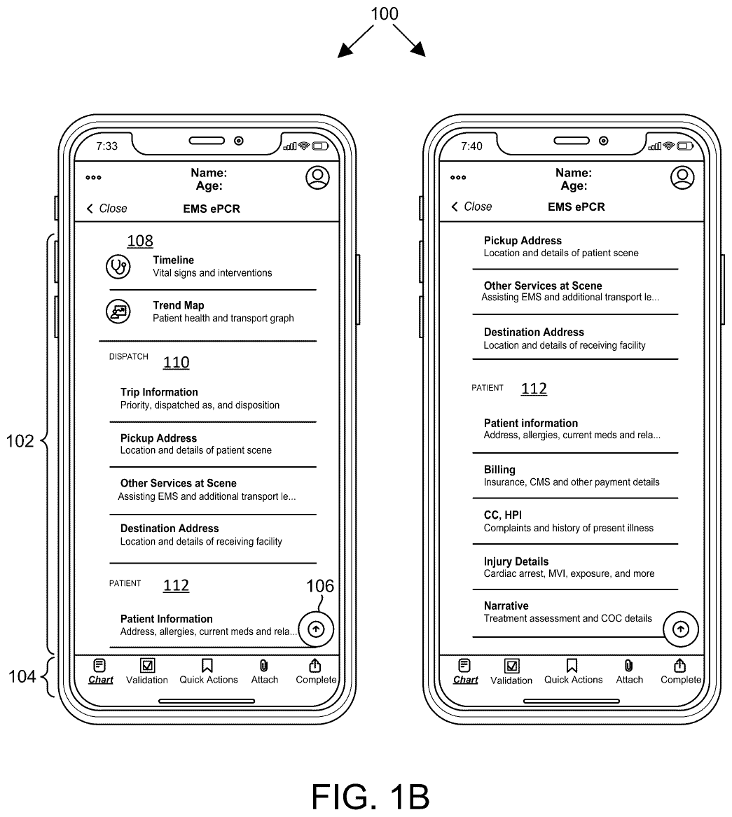

By way of introduction, FIGS. 1A and 1B illustrate screens of an ePCR user interface (UI) implemented by a mobile EMS charting application in some examples. As can be seen in review of FIGS. 1A and 1B, the design of the mobile EMS charting application reflects a mobile-first philosophy. In addition, the mobile EMS charting application leverages mobile device hardware and technology associated with a mobile computing device to collect and input data in an offline mode (e.g., when its host device is not connected to a network) and an online mode (when the host device is connected to a network). The mobile computing device, as the host device, can operate in the online mode and shift to operate in the offline mode and similarly operate in the offline mode and shift to operate in the online mode. Likewise, the mobile EMS charting application is provisioned with online and offline capabilities. In at least some examples, the mobile EMS charting application can validate data entered into ePCR data fields, during entry of the data, and complete or “lock” the ePCR while operating in the offline mode. Further, in at least some examples, the mobile EMS charting application is configured to tightly integrate, via the host device's web browsing capabilities, with a cloud-based mobile EMS charting application to receive configuration information from, and to submit ePCR data to, the cloud-based application.

In certain examples, the mobile EMS charting application is configured to control its host device to render and interact with a user via an ePCR user interface. In some implementations, the ePCR user interface includes a variety of UI screens that enable the mobile EMS charting application to receive user input specifying various ePCR data field values and/or requests to take other programmatic actions. These requests may include requests to navigate to a particular data set section of an ePCR and/or requests to lock the ePCR. Some examples of the UI screens included within one example of the ePCR user interface are illustrated in FIGS. 1A and 1B. The screens illustrated in FIGS. 1A and 1B support workflows routinely performed by users, such as EMS caregivers, in the field. FIG. 1A illustrates a login screen 114, a shift startup screen 120, and a chart selection screen 128.

According to one workflow, a user logs into the mobile EMS charting application at the start of a shift. The screen 114 supports this operation. As shown in FIG. 1A, the screen 114 includes security credential controls 116 and a login control 118. In this example, the mobile EMS charting application is configured to receive input specifying a user identifier and a password via the security controls 116 and to attempt authentication of a user associated with the user identifier and the password in response to receiving input selecting the login control 118. In some examples, to authenticate the user the mobile EMS charting application may interoperate with an identity provider that is local to the host device or implemented by an edge or cloud server in communication with the host device. Alternatively or additionally, the mobile EMS charting application may utilize one or more biometric sensors (e.g., a fingerprint scanner) embedded within the host device to authenticate the user.

Continuing with the example workflow, the user enters shift information including shift detail and crew member information. The shift startup screen 120 supports this operation. In some examples, the mobile EMS charting application is configured to control the host device to render the screen 120 if the attempt to authenticate the user is successful. As shown in FIG. 1A, the screen 120 includes shift detail controls 122, crew member controls 124, and a next screen control 126. The shift detail controls 122 include a shift control, a base control, a staffing level control, and a vehicle control. The crew member controls 124 include a crew member control and a role control. In this example, the mobile EMS charting application is configured to receive input specifying shift and crew member information via the controls 122 and 124. The mobile EMS charting application is further configured to store the shift and crew member information in a local data store. Further, in this example, the EMS charting device is configured to control the host device to render the chart selection screen 128 in response to receiving input selecting the next screen control 126.

Continuing with the example workflow, the user proceeds to enter ePCR data. To support this operation, in some examples, the mobile EMS charting application is configured to control the host device to render a new chart screen in response to receiving input selecting the add control 130 of the screen 128 illustrated in FIG. 1A. FIG. 1B illustrates one example of a new chart screen 100 rendered in some examples.

As shown in FIG. 1B, the screen 100 includes several section controls 102, a navigation bar 104, and a top control 106. Each of the section controls 102 may be referred to herein individually as a section control 102. The mobile EMS charting application is configured to detect user interaction with the host device via the screen 100, and to respond thereto. Such user interaction with the host device may take the form of one or more touchscreen gestures. Gesture input may include swipes, taps, pinches, shakes and other input generated by interaction of the human hand directly with a touchscreen or a device including the touchscreen. For instance, the mobile EMS charting application may be configured to detect a swipe (e.g., up or down) over the section controls 102 and to respond to the detected swipe by scrolling the section controls 102 in the direction of the swipe. The mobile EMS charting application may be further configured to detect selection (e.g., a touch or tap) of an individual section control 102 and to respond to the selection by opening a data set section of the ePCR associated with the individual section control 102. Similarly, the mobile EMS charting application may be configured to detect selection of a control within the navigation bar 104 and to respond to the selection by opening a screen associated with the selected control. Further, in some examples, the mobile EMS charting application is configured to detect selection of the top control 106 and to respond to the selection by scrolling the section controls 102 downward until the top of the screen 100 is displayed.

Continuing with the screen 100, the section controls 102 are arranged into groups to enable the user to locate and navigate easily to various sections of the new ePCR. In the example shown, these control groups include an overall control group 108, a dispatch group 110, and a patient group 112. For instance, the section controls 102 within the control group 108 enable a user to navigate to screens that access patient encounter timeline information and trend map information. The timeline information may include timestamps of common EMS call milestones, such as dispatch time, arrival time, times at which specific interventions are administered and the like. The trend map information may indicate patient condition (e.g., vital signs) as recorded at each of the milestones. The section controls 102 within the dispatch group 110 enable a user to navigate to screens that access trip information, pickup address information, destination address information, and information regarding other services at the scene. The trip information may include dispatch priority, type of service, and unit number for an EMS vehicle traveling to a patient encounter. The pickup address information may include a location and access details of a location of a patient. The destination address information may include a location and access details of a facility targeted to receive the patient at the completion of the EMS call. The other services information may include identifiers of other EMS crews and transports involved in the EMS call. The section controls 102 within the patient group 112 enable a user to navigate to screens for various data set sections, for example, screens that access patient information, billing information, chief complaint information, injury information, and narrative information. The patient information may include patient address, allergies, current medications, and demographic information. The billing information may include insurance information, Medicare/Medicaid information, supplemental insurance information, and other information related to payment. The chief complaint information may include an indication of one or more complaints of the patient (e.g., symptoms, etc.), information regarding a history of the present illness, and the like. The injury information may include information regarding source of injuries sustained by the patient (e.g., cardiac condition, motor vehicle incident, exposure, etc.). The narrative information is a semi-structured or free-form assessment of the EMS call and typically addresses the patient's condition, medical history, interventions performed, results thereof, and other significant information regarding the EMS call.

In some examples, the mobile EMS charting application is configured to validate ePCR data entered via the screens described above both locally (e.g., without involving devices other than the host) and integrally with ePCR data entry. By avoiding batch validation of ePCR data as a separate activity, the mobile EMS charting application increases EMS caregiver efficiency in completing ePCRs while ensuring the ePCRs are complete and comply with all applicable rules. Selected features of various architectures that enable the mobile EMS charting application to implement this functionality will now be described.

### EMS Charting System

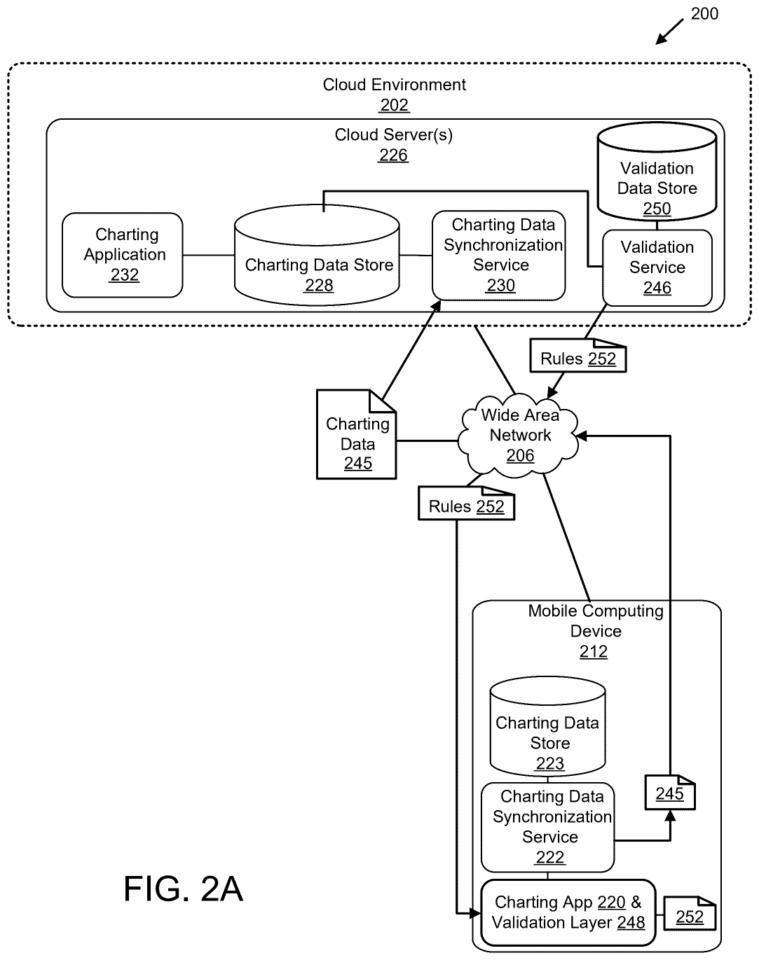

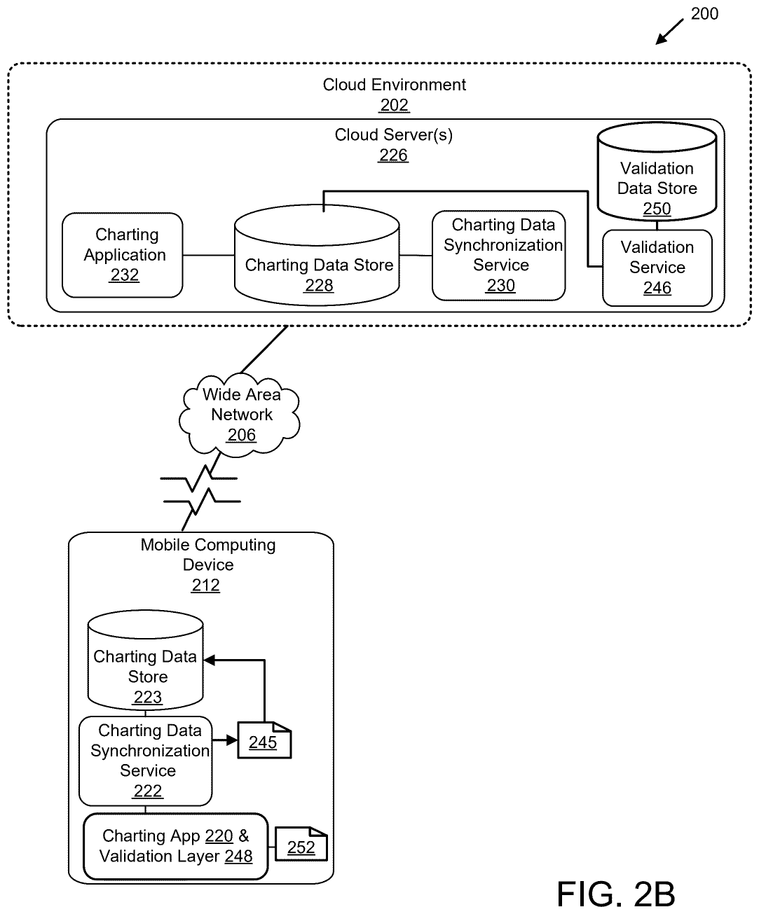

FIGS. 2A and 2B illustrate example EMS charting system implementations in accordance with some examples. As shown in FIGS. 2A and 2B, the EMS charting system 200 includes a cloud environment 202, a mobile computing device 212, and a wide area network 206 through which computing devices in the cloud environment 202 and the mobile computing device 212 can communicate and interoperate with one another. In some examples, the cloud environment 202 includes one or more cloud server(s) 226 that host a cloud EMS charting application 232, a charting data store 228, a validation service 246, and a validation data store 250. In some implementations, the cloud environment 202 may include a charting data synchronization service 230. The clouds server(s) 226 can include one or more physical and/or virtual servers.

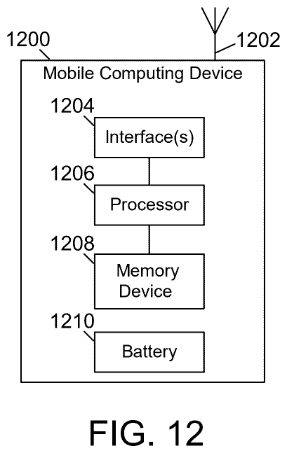

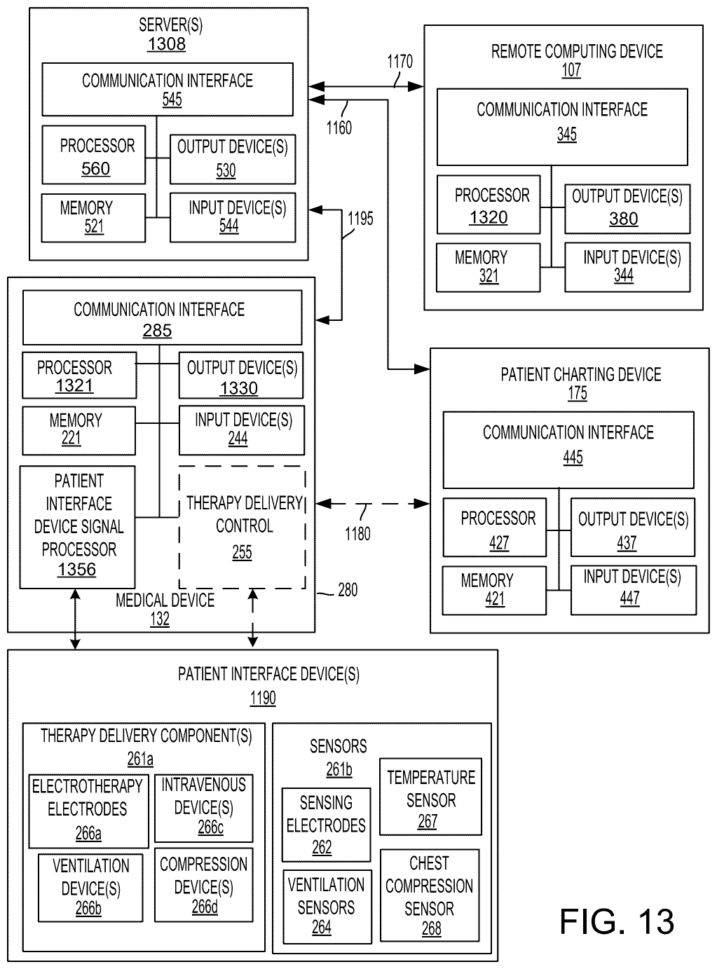

Continuing with examples illustrated by FIGS. 2A and 2B, the device 212 may be any mobile computing device, such as a smartphone, tablet computer, laptop computer, or other mobile computing device. Particular examples of the device 212 are described further below with reference to FIGS. 12 AND 13. As shown in FIGS. 2A and 2B, the device 212 is provisioned with a mobile EMS charting application 220 and a charting data store 223. In some implementations, the device 212 may include a charting data synchronization service 222. The application 220 may be implemented as a stand-alone, native app configured to run under the operating system of its host device. Alternatively, or additionally, the application 220 may be served to its host device as a browser-enabled application from the cloud EMS charting application 232. The application 220 may be configured to interact with a user to receive, validate, and store ePCR data values through a UI including a set of screens, such as the screens illustrated in FIGS. 1A and 1B above. Individual screens within the UI provided by the application 220 may include one or more user interface controls that are configured to receive input and/or display output. Some of these controls may be associated with one or more data fields of an ePCR that are sized and typed to store certain ePCR data values. The application 220 may be configured to receive input selecting the user interface controls and to execute, in response to receiving a selection of any given control, a process associated with the control. Such processes may be used for a variety of purposes including validation of ePCR data values input via one or more controls.

For instance, in some examples, the application 220 may include and/or interoperate with a validation layer 248 that is configured to validate ePCR data values locally (e.g., without resorting to computing resources outside of the mobile computing device 212). In some examples, this local validation may be contemporaneous with and a part of reception of ePCR data values via user input. As such, the local validation may be executed iteratively (e.g., each time new data is entered) on an ePCR field-by-field basis with the layer 248 updating indications of validation results each time a data value is entered into an ePCR data field. In this way, the layer 248 may iteratively update the indications of validations results. Moreover, in some examples, reception of ePCR data values and local validation thereof may be executed regardless of whether the device 212 is connected or disconnected from the network 206. It should be noted that indications of validation results may include indications of validation errors or indications of successful validation. An indication of successful validation may include, for example, omission or removal of an indication of a validation error.

In some examples in accordance with FIGS. 2A and 2B, the layer 248 receives an electronic validation file specifying a set of rules 252 from the validation service 246 via the network 206 prior to validation activity. For instance, in some examples, the layer 248 requests and receives the electronic validation file specifying the rule set 252 as part of initial installation of the application 220 on the device 212. This installation may be performed by an installation program that is part of the system 200. The electronic validation file may be communicated to the layer 248 via a variety of communication methods and protocols, such as file transfer protocols, streaming, and the like. Alternatively or additionally, in certain examples, the layer 248 receives a file specifying an updated rule set 252 periodically, in response to a request for the same, and/or as part of an unsolicited rules update process executed by the service 246. In some implementations, the layer 248 is configured to respond to reception of the file specifying the rule set 252 by parsing the file to extract the rule set 252 and/or storing a local copy of the file or the rule set 252 in a local data store. As shown in FIGS. 2A and 2B, the set of rules 252 is stored in an electronic validation file, but other suitable data stores (e.g., a database) will be apparent in light of this disclosure.

In some examples, the application 220 is configured to receive input specifying a request to access an ePCR via a screen, such as the screen 100 described above with reference to FIG. 1B. In these examples, the application 220 is further configured to respond to such a request by controlling the device 212 hosting the application 220 to retrieve and execute code that implements the screen. This code may be stored in, and retrieved from, memory incorporated within the device 212. In certain examples, the code retrieve may include all, or a portion of, the layer 248 and/or the locally stored rule set 252. The application 220 may be further configured to receive input that specifies data values for ePCR data fields via user interface controls that are associated with the ePCR data fields. These data values may take a variety of forms, such as text, numbers, alphanumeric values, enumerated values, and/or strings, among other values.

In some implementations, the layer 248 is configured to test ePCR data values and generate validation results that denote whether the ePCR data values are valid (e.g., in compliance with the rule set 252) or invalid (e.g., not in compliance with the rule set 252). This validity testing may include, for example, comparing the data values to a set of enumerated values specified by a rule; comparing the data values to a range of values specified by a rule; comparing the data values to string lengths specified by a rule; ensuring required ePCR data fields are populated with a data value, and the like. In certain examples, individual rules may specify an error message to be displayed if the rules are violated. This message may be specified, for example, by an EMS agency. In some examples, the validity testing specified by the rule set 252, and executed by the layer 248, is defined by a set of standardized validation rules, such as a set of rules required to comply with the National Emergency Medical Services (NEMSIS) standard. Further, in certain examples, the validity testing specified by the rule set 252 can be defined by other standards, such as governmental and/or EMS agency-specific standards. Other validity testing operations will be apparent in view of this disclosure.

In some examples, the layer 248 is configured to determine whether a given rule is applicable with reference to a variety of information. For instance, in some examples, the rule set may include hierarchical information that specifies which rules are applicable along various dimensions. For instance, in at least one example, the hierarchical information specifies tiers of rules that are applicable to particular countries, states, regions, agencies, facilities, EMS service types, and EMS call types. As such, in some examples, the layer 248 may determine whether a particular rule is applicable based on the ePCR data field associated with a user interface control being accessed, an authenticated identity of a current user of the application 220, a geolocation of the device hosting the validation layer, and/or the hierarchical information included in the rule. Moreover, some rules may include logical implications to be evaluated to determine whether the rule is applicable and assertions to be evaluated regarding data values. It should be appreciated that the applicability of some rules may depend on multiple data values stored in multiple ePCR data fields. For example, a cross state transfer rule may apply only where the pick-up location and the destination location are in different states. As another example, a second rule may apply only if a first, applicable rule is determined to be valid.

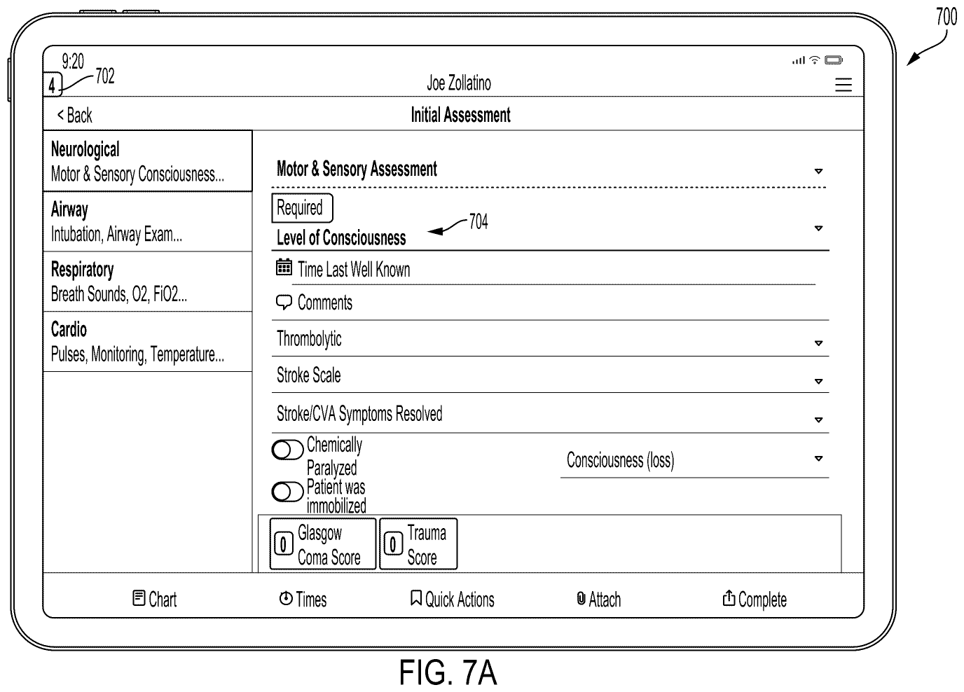

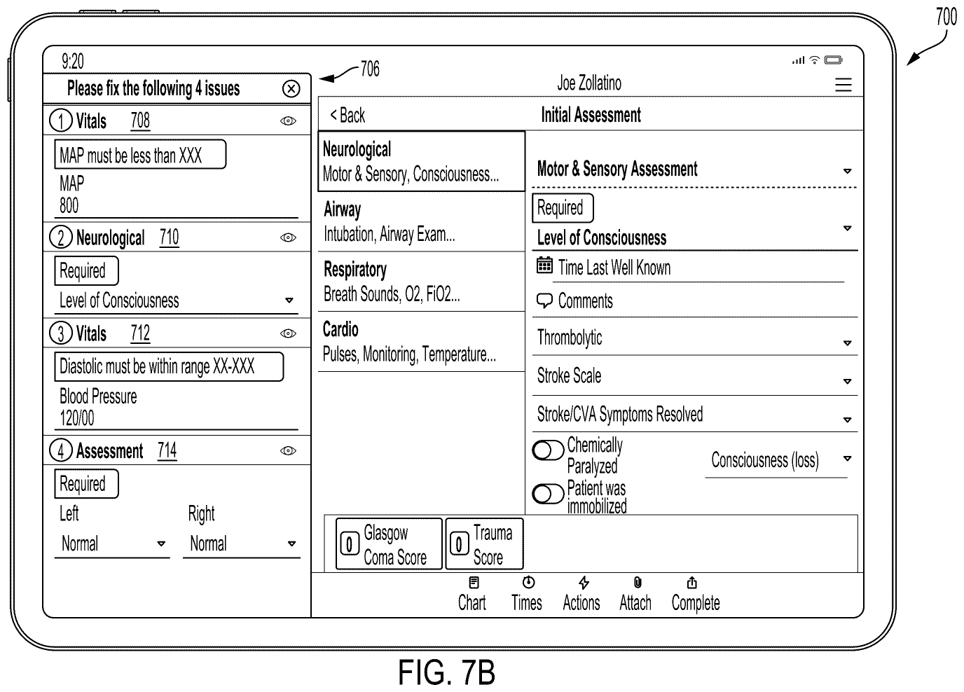

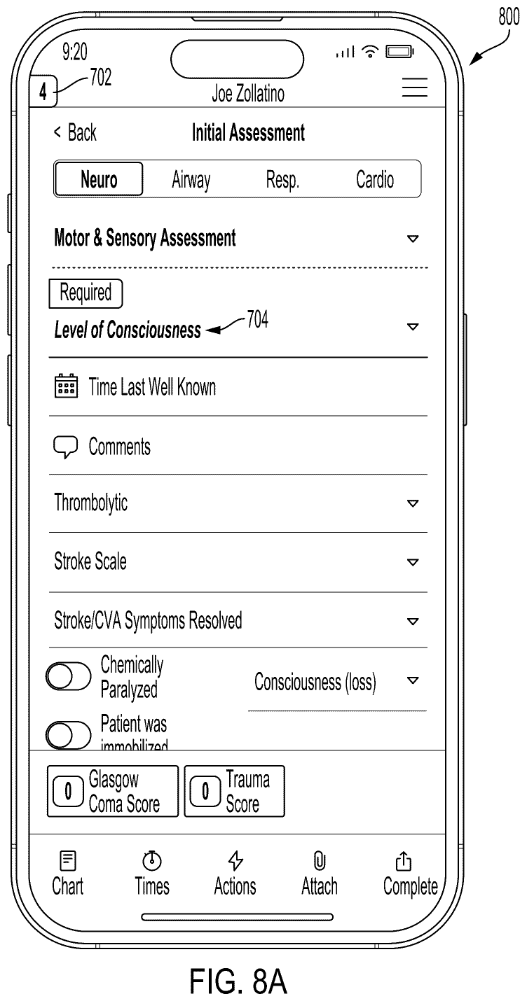

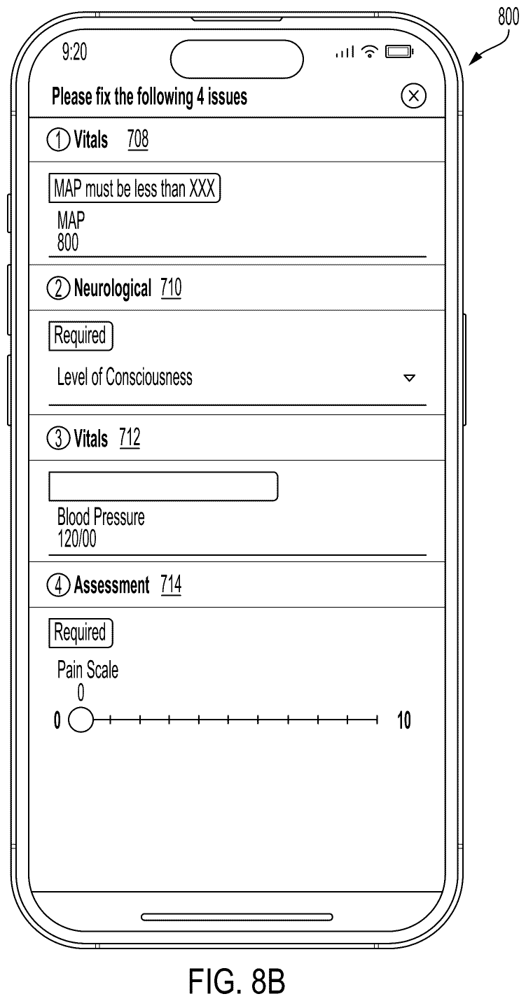

In some examples, the application 220 is configured to display indications of validation results (e.g., indications of validation errors and/or indications of successful validation) in the user interface. For instance, in certain examples, the application 220 controls its host device to display these indications via user interface controls that are associated with the ePCR data fields and data values. In some examples, the application 220 is configured to display the indications immediately and/or on a field-by-field basis (e.g., before the host transitions focus of the UI between user interface controls). Alternatively or additionally, in some examples, the application 220 displays the indications within a control group dedicated to display of validation results. This control group can reside in a screen distinct from other screens of the UI and/or be overlaid upon the other screens (e.g., within a panel). Examples of particular UI features through which indications of validation results can be displayed are described further below with reference to FIGS. 7A-8B.

In some examples, the service 246 is configured to create and communicate the rule set 252 to the layer 248 via the network 206. For instance, in some examples the service 246 is configured to create the rule set 252 by transforming a set of rules (e.g., NEMSIS compliance rules) from a first syntax to a second set of rules (e.g., the rule set 252) that adheres to a second syntax different from the first syntax. This transformation can increase the speed with which ePCR data is subsequently validated or otherwise certified to be in accordance with the rule set 252, thus enabling field-by-field validation with acceptable or non-noticeable latency. For instance, in certain examples, the rule set 252 can be encoded in a syntax that is directly or natively translatable by a runtime engine and/or executable by a real or virtual machine, thus enabling a rule validation architecture that is simpler than an architecture that is required to apply rule sets with differing and/or non-executable syntaxes. Although the first syntax and the second syntax may comply with any of a variety of formats, in at least one example, the first syntax is a Schematron syntax (e.g., Schematron V3) and the second syntax is a JAVASCRIPT syntax (e.g., EMCAScript 2023). For instance, in some examples, the rule set 252 is encoded as a JAVASCRIPT library can be linked to or otherwise make available for execution within a browser container of the application 220. As such, in these examples, the second syntax (e.g., JAVASCRIPT) is executable by the execution environment (e.g., the browser container). In these examples, the application 220 may be a hybrid mobile application. Other execution environments for the application 220, the layer 248, and/or the rule set 252 will be apparent. In the system, the first syntax may be a Schematron syntax, or other syntax based on a rule-based validation language. However, Schematron is different from extensible mark-up language (XML) and may not be interechangeable therewith. In some cases, Schematron may provide functionality unavailable from another rule-based validation language. The second syntax may be a JAVASCRIPT syntax, or other syntax associated with a programming language compatible with (for example, capable of expressing executable rules translated, e.g., transpiled, from) the first syntax (for example, either with Schematron, or with another rule-based validation language).

In certain implementations, the data store 250 is configured to house both the rule set 252 and the set of rules transformed by the service 246. In some examples, the service 246 is configured to retrieve the rule set 252 from the data store 250 and transmit the rule set 252 (e.g., within a JAVASCRIPT Object Notation (JSON) file) to the layer 248 in response to occurrence of an event, such as expiration of a timer, receipt of a message from the layer 248 requesting a current version of the rule set 252, or generation of a new rule set 252.

In some implementations, the synchronization service 230 and the synchronization service 222 are configured to interoperate with one another (e.g., via a set of application programming interface (API) calls) to maintain a common view of the ePCR data stored within the EMS charting system 200 for the application 220 and the application 232. In certain examples, the set of API calls allow other processes to access ePCR data stored in the data stores 228 and 223 via the services 230 and 222. In some examples, when processing these API calls, the services 230 and 222 transmit, receive, and process synchronization messages that indicate each change made to ePCR data, a timestamp of the change, and an identifier of the originator of the change (e.g., an automated process, a user, etc.).

The APIs exposed by the services 230 and 222 may be implemented using a variety of interoperability standards and architectural styles. For instance, in one example, the APIs include a web services interface implemented using a representational state transfer (REST) architectural style. In this example, the API communications are encoded in Hypertext Transfer Protocol (HTTP) along with JSON and/or extensible markup language. In some examples, portions of the HTTP communications may be encrypted to increase security. Alternatively or additionally, in some examples, at least one of the APIs is implemented as a .NET web API that responds to HTTP posts to particular URLs with data descriptive of ePCR data stored in the data store 228 and/or the data store 223. Alternatively or additionally, in some examples, at least one of the APIs is implemented using simple file transfer protocol commands and/or a proprietary application protocol accessible via a TCP socket. Thus, the APIs as described herein are not limited to a particular implementation.

As described above, in certain examples, the service 222 is configured to maintain the data store 223 in local storage for use by the application 220. In some of these examples, the service 222 can incorporate a service worker (or some other monitoring process executing on a thread distinct from a thread executing the application 220) configured to monitor and recognize a network connectivity status between the device 212 and the network 206. When the connection to the network 206 is active (e.g., a network interface of the device 212 is in a connected state or state of connection) and the service 222 is operating in an online mode, the service 222 exchanges synchronization messages with the service 230. These synchronization messages may specify, for example, ePCR data values indicated as the charting data 245 in FIG. 2A. However, when the connection to the network 206 is inactive (e.g., a network interface of the device 212 is in a disconnected state or state of disconnection) and the service 222 is operating in an offline mode, the service 222 cashes synchronization messages addressed to the service 230 and subsequently transmits the synchronization messages when the connection to the network 206 resumes. For instance, in some examples, the service 222 is configured to wait for a timeout period after detecting that the network interface is in a state of disconnection before attempting to establish (or re-establish) a connection to the network 206. After detecting that the network interface is connected to the network, the service 222 transmits the cached synchronization messages. These messages may include, for example, results of the validation processes described herein. FIG. 2B illustrates an example of this situation in which the synchronization messages, which specify ePCR data values indicated as the charting data 245, are cached within the data store 223. It should be noted that when a network interface is in a state of disconnection, the service 222 and other processes executed by the host device are forced to rely on resources local to the host device. As such, when the network is in a state of disconnection, remote computing devices, such as cloud servers, are not accessible or otherwise available to the host device or its processes, at least temporarily.

In certain implementations, the data store 228 is configured to store ePCR data for manipulation by the application 232 and/or the service 230. Playing a similar role in certain examples, the data store 223 is configured to store ePCR data for local manipulation by the application 220 and/or the service 222. Each of the data stores 228 and 223 may be organized according to a variety of physical and/or logical structures. In at least one example, the data store 228 and/or the data store 223 are implemented within a relational database having a highly normalized schema and accessible via a structured query language (SQL) engine, such as ORACLE or SQL-SERVER. In some examples, at least portions of the data stores 228 and 223 are implemented as flat operating system files including serialized, proprietary data structures. Thus, the data stores 228 and 223 as described herein are not limited to a particular implementation.

In some examples illustrated by FIGS. 2A and 2B, the network 206 may include any communication network through which devices in the cloud environment 202 and the device 212 can exchange data. In some examples, the network 206 includes and supports wireless network and/or wired connections. For instance, in these examples, the network 206 can support one or more networking standards such as WI-FI, GSM, CDMA, USB, BLUETOOTH, CAN, ZIGBEE, Wireless Ethernet, Ethernet, and Transmission Control Protocol (TCP)/Internet Protocol (IP), among others. In at least one example, the network 206 includes both private networks, such as local area networks, public networks, such as the Internet, and cellular networks. It should be noted that, in some examples, the network 206 includes one or more intermediate devices involved in the routing of packets from one endpoint to another. However, in other examples, the network 206 involves only two endpoint devices that each have a network connection directly with the other.

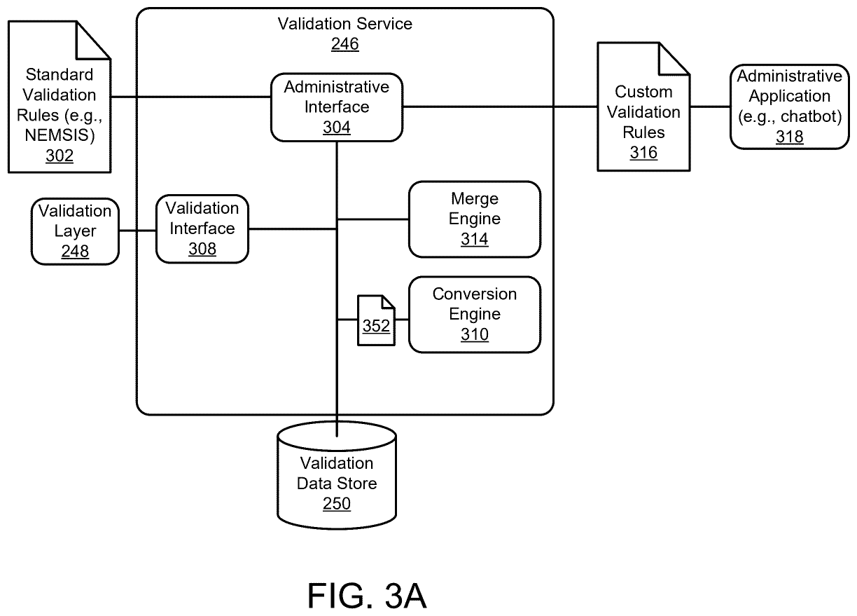

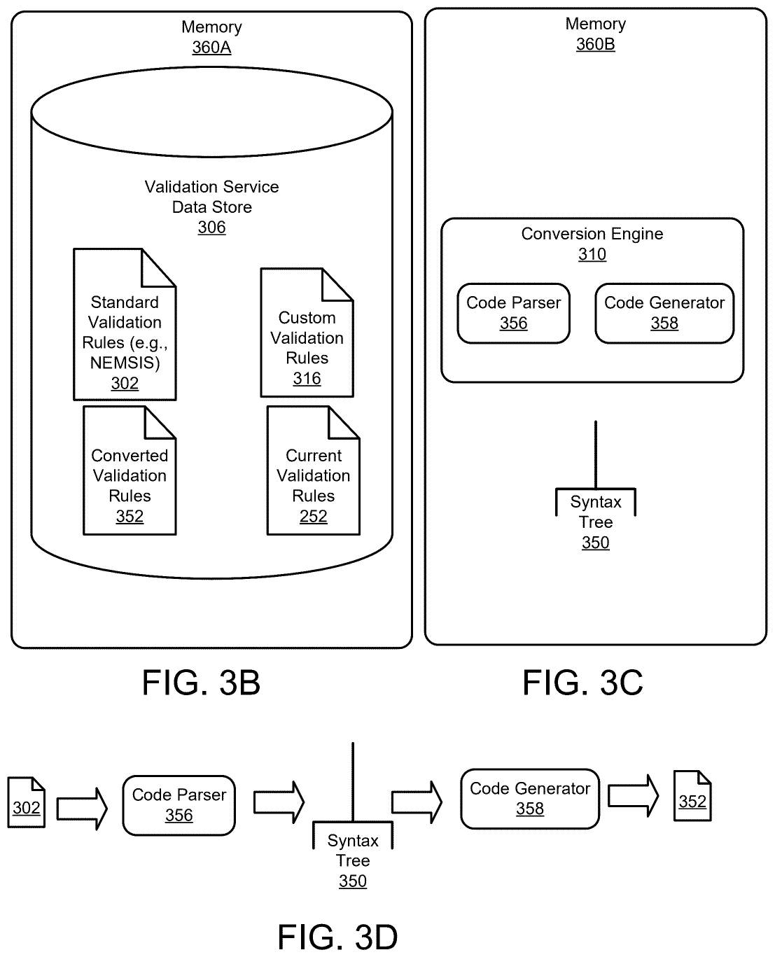

Turning now to FIG. 3A, one implementation of the validation service 246 introduced in FIGS. 2A and 2B is illustrated. As shown in FIG. 3A, the service 246 includes an administrative interface 304, a validation layer interface 308, a merge engine 314, and a conversion engine 310. The interface 304 is further configured to operate on a set of standard validation rules 302 (e.g., a first set of rules) and, if present, a set of custom validation rules 316 (e.g., a second set of rules) that may be generated by an administrative application 318. The custom validation rules may specify policies of a healthcare provider (e.g., aspirin should be offered to all cardiac patients) with which EMS personnel are required to comply. The interfaces 308 and 304 and the engines 314 and 310 are configured to communicate with the validation data store 250 introduced in FIGS. 2A and 2B. The interface 308 is configured to communicate with the validation layer 248.

As shown in FIG. 3A, the administrative interface 304 is configured to expose and implement an API to receive and process electronic files specifying the set of standard validation rules 302 and the set of custom validation rules 316. This processing may include parsing the files to extract the rule sets 302 and 316 and store the rule sets 302 and 316 in the data store 250. For instance, in some examples, the files specifying the rule sets 302 and 316 are JSON files although other types of files are contemplated. An example portion of a JSON file that specifies two rules follows.

```
{
  “rules”: [
    {
      “id”: “Rule_769”,
      “name”: “Rule_769”,
      “description”: “Rule_769”,
      “script”: “function rule769( ) {if(eVitals07 !== ‘’ && eVitals06
!= ‘’ && number (eVitals07) > number(eVitals06)) {return ‘DBP
(Diastolic Blood Pressure) Cannot Be Greater than Systolic.’}}”
    },
      {
      “id”: “Rule_186”,
      “name”: “Rule_186”,
      “description”: “Rule_186”,
      “script”: “function rule186( ) {if(eDisposition30 === ‘’ &&
eDisposition27 === 4227001) {return ‘eDisposition.30 should be
recorded when eDisposition.27 is Patient Contact Made’}}”
    }
  ]
}
```

In some examples, the interface 304 is configured to receive and process communications from the engine 314 that specify errors detected during execution of merge and/or test processes involving the rule set 316. In these examples, the interface 304 may communicate the errors to the application 318 to notify a user of the error encountered.

Continuing with the example of FIG. 3A, the conversion engine 310 is configured to retrieve a rule set (e.g., the rule set 302 and/or the rule set 316) from the data store 250, convert the rule set from a first syntax to a second syntax, and store the converted rule set as a rule set 352. In some examples, the first syntax is Schematron and the second syntax is JAVASCRIPT, although other potential first and second syntaxes will be apparent in view of this disclosure. One example of a rule encoded in a Schematron syntax follows.

```
<sch:pattern>
  <sch:title>eVitals.07 related rule.</sch:title>
  <sch:rule
context=“nem:PatientCareReport/nem:eVitals/nem:eVitals.VitalGroup/ne
m:eVitals.BloodPressureGroup”>
      <sch:let name=“nemsisElements” value=“nem:eVitals.07”/>
        <sch:assert
          id=“Rule_769”
          role=“[ERROR]”
          diagnostics=“nemsisDiagnostic”
          test=“not (
            nem:eVitals.07 != ‘’ and
            nem:eVitals.06 != ‘’ and
            number (nem:eVitals.07) &gt; number(nem:eVitals.06)
          )”>
              DBP (Diastolic Blood Pressure) Cannot Be Greater than
Systolic.</sch:assert>
    </sch:rule>
  </sch:pattern>
```

Continuing with this example, the rule encoded in a JAVASCRIPT syntax follows.

```
function rule769 ( )
{
  if (
        (eVitals07 !== ‘’) &&
        (eVitals06 !== ‘’) &&
        number(eVitals07) > number(eVitals06)
      )
  {
    return “DBP (Diastolic Blood Pressure) Cannot Be Greater than
Systolic.”
  }
}
```

One example of selected portions of the conversion engine 310 is described further below with reference to FIG. 3C. In some examples, the engine 310 is configured to test the rule set 352 by processing a set of test data using the rule set 352. In these examples, the engine 310 is configured to deem the conversion to be successful if no errors are detected. In certain examples, the engine 310 is configured to to communicate any errors encountered to the interface 304, which may relay the errors to an external system, such as the application 318, to notify a user of the error.

Continuing with the example of FIG. 3A, the engine 314 is configured determine whether the rule set 316 is available within the data store 250, and, if so, retrieve the rule sets 352 and 316 from the data store 250 and merge them into the rule set 252 of FIGS. 2A and 2B. In some examples, the engine 314 is configured to scan both rule sets 352 and 316 for code that refers to common ePCR data fields and compare all constraints placed on the common ePCR data fields for conflicts. For instance, a first rule that requires an ePCR data value to be within a first range conflicts with a second rule that requires the ePCR data value to be within a second range if the first range and the second range do not at least partially overlap. Other types of potential conflicts will be apparent in view of this disclosure. Further, in these examples, the engine 314 is configured to deem the merge to be successful if no conflicts are identified and store the combined code base as the rule set 252 in the data store 250. Alternatively or additionally, in some examples, the engine 314 is configured to combine the rule sets 352 and 316 into a single code base and test the combination by processing a set of test data using the combined code base. In these examples, the engine 314 is configured to deem the merge to be successful if no errors are detected and store the combined code base as the rule set 252 in the data store 250. Additionally or alternatively, in certain examples, the engine 314 is configured to test the rule set 352, as described herein, even if no rule set 316 is available. In these examples, the engine 314 is configured to store the rule set 352 as the rule set 252 in the data store 250 if testing is successful. Regardless of the specific merge and test processes executed, in some examples, the engine 314 is configured to communicate any errors encountered to the interface 304, which may relay the errors to an external system, such as the application 318, to notify a user of the error. It should also be noted that, in certain examples, the rule sets 352 and 316 share a common syntax to facilitate their combination. In some examples, the engine 314 may interoperate with the engine 310 to convert the rule set 316 from a first syntax (e.g., Schematron) to a second syntax (e.g., JAVASCRIPT).

Continuing with the example of FIG. 3A, the interface 308 is configured to retrieve rule data sets (e.g., the rule set 252) from the data store 250 and to expose and implement an API to communicate the retrieved rule data sets to the layer 248. These retrieval and communication functions may be triggered by an API call from the layer 248 or unsolicited and in response to an event detected by the interface 308 (e.g., publication of a new rule set 252). In some examples, the interface 308 is configured to generate a JSON file specifying the rule set 252 and transmit the rule set 252 within the file to the layer 248 via, for example, an HTTP message. Once the layer 248 receives and stores a local copy of the file specifying the rule set 252, the layer 248 can locally validate ePCR data values using the local copy, even if the device hosting the layer 248 is operating in an offline mode.

Continuing with the example of FIG. 3A, the application 318 is configured to interact with a user to generate the rules set 316, communicate the rules set to the interface 304 via one or more API calls, and receive and process responses thereto. For instance, in some examples, the application 318 controls its host device to display a UI including one or more screens with controls configured to receive input specifying custom rules. The input that these controls are configured to accept may include text, dropdowns populated by enumerated values, and the like. In some examples, the screens may include controls configured to display indications of errors encountered during merge and/or test processes executed by the engine 314. Alternatively or additionally, in some examples, the application 318 is configured to control its host device to implement a chatbot that receives and responds to human language. Other implementations of the application 318 will be apparent in view of this disclosure.

Turning now to FIG. 3B, a memory 360A storing one example of the data store 250 introduced in FIGS. 2A and 2B (a validation service data store 306) is illustrated. The memory 360A may be volatile memory, non-volatile memory or a combination thereof. In at least one example, the memory 360A is implemented as a memory device, such as the memory device 1208 described further below with reference to FIG. 12. As shown in FIG. 3B, the data store 306 includes the current rule set 252 introduced in FIG. 2A; and the rule sets 302, 316, and 352 introduced in FIG. 3A.

Turning now to FIG. 3C, a memory 360B storing one example of the conversion engine 310 introduced in FIG. 3A is illustrated. Like the memory 360A, the memory 360B may be volatile memory, non-volatile memory or a combination thereof and may be implemented as a memory device, such as the memory device 1208. In some examples, non-volatile memory may be referred to as persistent memory. In an implementation, memory 360A may be non-volatile memory and memory 360B may be volatile memory. Similarly and alternatively, memory 360A may be volatile memory and memory 360B may be non-volatile memory. Alternatively, memories 360A and 360B may both be volatile memory or may both be non-volatile memory. As further shown in FIG. 3C, in some examples the memory 360B stores a syntax tree 350. In these examples, the syntax tree 350 is a data structure that stores a tokenized version of the rule set 302. As such, the syntax tree 350 may be an intermediate representation of the rule set 302 that is organized into a structure that can be parsed for generation of the rule set 352 (e.g., in a second syntax different from the first syntax of the rule set 302). As illustrated in FIG. 3C, the conversion engine 310 includes a code parser 356 and a code generator 358. In some examples, the code parser 356 is configured to tokenize the rule set 302 into a set of tokens specific to the first syntax and store the tokens in the syntax tree 350. Further, in these examples, the code generator 358 is configured to parse the syntax tree 350 (e.g., read the tokens therefrom) and generate the rule set 352 in the second syntax. This processing pipeline is illustrated in FIG. 3D and may be referred to herein as “transpiling.” In some examples, the first syntax is Schematron and the second syntax is JAVASCRIPT. However, the examples described herein are not limited to Schematron and JAVASCRIPT. In another example, the first syntax is Schematron and the second syntax is object code. In another example, the first syntax is an XML-based syntax other than Schematron and the second syntax is Python. Other examples will be apparent in light of this disclosure. It should be noted that, in some examples, the converted rule set 352 and the charting application 220 share a common syntax that is executable by the same execution environment (e.g., operating system, virtual machine, runtime layer, browser container, etc.). It should also be noted that, in some examples, syntax in which the rule set 302 is encoded is not executable by the execution environment and is, therefore, incompatible with local validation.

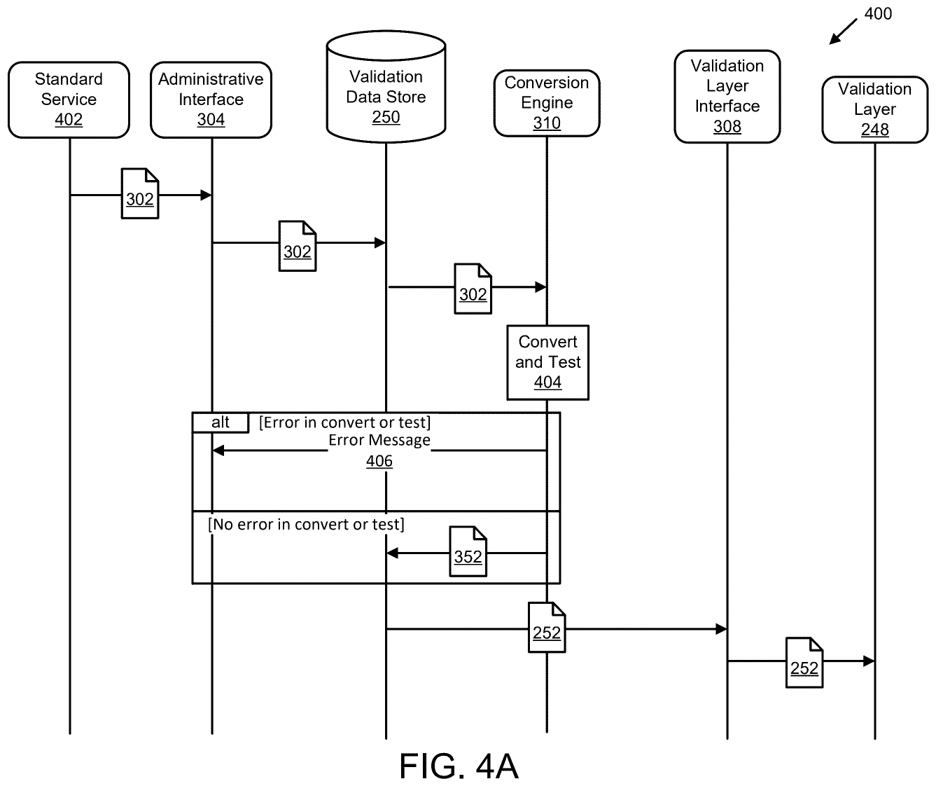

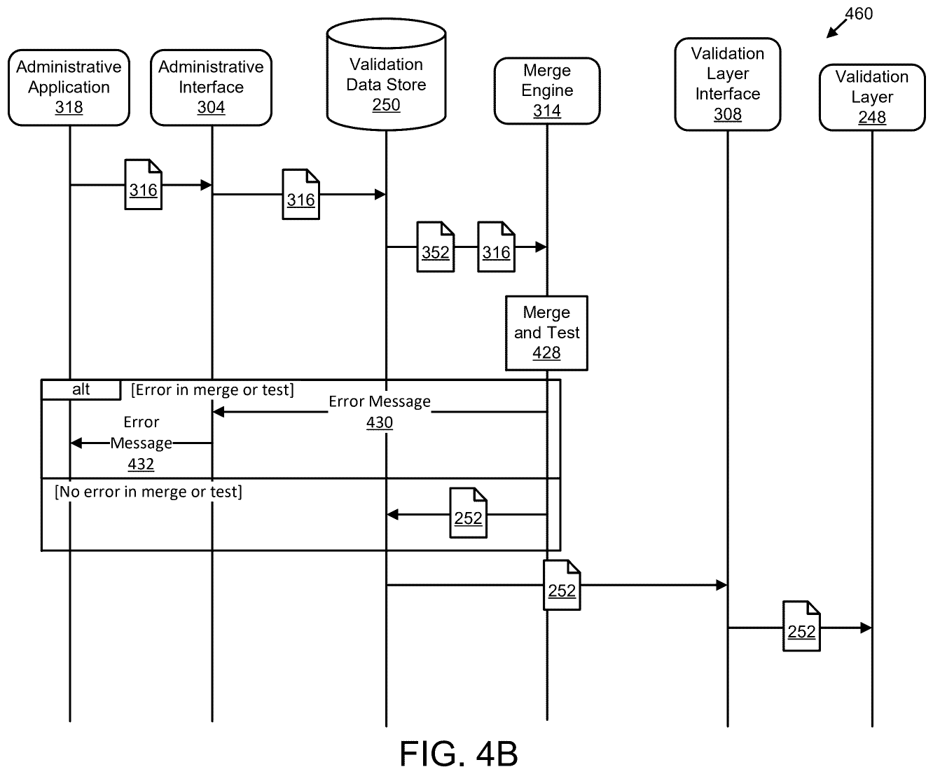

Turning now to FIG. 4A, a process 400 of generating and distributing rule sets is illustrated. In some examples, the process 400 may be executed by an EMS charting system (e.g., the EMS charting system 200 introduced in FIGS. 2A and 2B). More specifically, portions of the process 400 may be executed by the administrative interface 304, the conversion engine 310, and the validation layer interface 308 introduced in FIG. 3A. Additional portions of the process 400 may be executed by the validation layer 248 introduced in FIG. 2A. The engine 310 and the interfaces 304 and 308 may interoperate with the validation data store 250 introduced in FIG. 2A.

As shown in FIG. 4A, the process 400 starts with a standard service 402 releasing a rule set 302 for public use. For instance, in some examples, a standards body (e.g., NEMSIS) makes a set of compliance rules available from a uniform resource locator (URL) of record, and the interface 304 downloads the rule set 302 from the URL. Upon completion of the download, the interface 304 stores the downloaded rule set 302 in the data store 250.

Continuing with the process 400, the engine 310 retrieves the rule set 302 from the data store 250. The engine 310 converts and tests 404 the rule set 302 to generate the rule set 352 introduced in FIG. 3A. In some examples, tests executed by the engine 310 can include syntax checking to ensure the rule set 352 is properly formed and may further include checking portions of the rule set 352 against test data previously established as being correct. If the engine 310 determines that the conversion or test were unsuccessful, the engine 310 generates a message 406 identifying the error and communicates the message 406 to the interface 304. In some examples, the interface 304 may interoperate with an administrative application (e.g. the administrative application 318 introduced in FIG. 3A) to further handle and resolve the error. If the engine 310 determines that the conversion and test were successful, the engine 310 stores the rule set 352 in the data store 250. In some examples, the engine 310 stores the rule set 352 in association with an indication that the rule set 352 is the current rule set 252, introduced in FIG. 2A, that is to be distributed to validation layers in the field (e.g., the layer 248).

Continuing with the process 400, the interface 308 retrieves the rules set 252 from the data store 250 and communicates the rules set 252 to the layer 248. For instance, in one example, the interface 308 responds to a periodic HTTP post from the layer 248 with a JSON file specifying the rule set 252. Other communication methods will be apparent in view of this disclosure.

Continuing with the process 400, the layer 248 processes the rules set 252. For instance, in one example, the layer 248 parses a JSON file specifying the rule set 252 and stores the rules set 252 locally for subsequent validation processing as described herein.

Turning now to FIG. 4B, a process 460 of generating and distributing rule sets is illustrated. In some examples, the process 460 may be executed by an EMS charting system (e.g., the EMS charting system 200 introduced in FIGS. 2A and 2B). More specifically, portions of the process 460 may be executed by the administrative application 318, the administrative interface 304, the merge engine 314, and the validation layer interface 308 introduced in FIG. 3A. Additional portions of the process 460 may be executed by the validation layer 248 introduced in FIG. 2A. The engine 314 and the interfaces 304 and 308 may interoperate with the validation data store 250 introduced in FIG. 2A. In this example, the rule set 352 introduced in FIG. 3A has been previously stored in the data store 250, for example by execution of the process 400 and/or 450.

As shown in FIG. 4B, the process 460 starts with the application 318 communicating a rule set 316 to the interface 304. For instance, in some examples, the application 318 transmits an electronic file specifying the rule set 316 to the interface 304 in response to receiving input from a user requesting the same. In certain examples, the rule set 316 is encoded in a second syntax that is shared with the rule set 352, although this is not a requirement. In examples where the rule set 316 is encoded in a first syntax different from the second syntax of the rule set 352, the service 246 may convert the rule set 316 from the first syntax to the second syntax the various processes described herein. Subsequent to receipt and any necessary conversion, the interface 304 stores the rule set 316 in the data store 250.

Continuing with the process 460, the engine 314 retrieves the rule set 316 and the rule set 352 from the data store 250. The engine 314 merges and tests 428 the rule sets 316 and 352 to generate the rule set 252 introduced in FIG. 2A and determines whether the merge and test operations were successful or unsuccessful. For instance, in some examples, the engine 314 determines whether any conflicts exist between individual rules and, if so, records a merge error. Examples of conflicting rules may include a first rule requiring an ePCR data field be populated with a data value from a first range and a second rule requiring that the ePCR data field be populated with a data value from a second range that does not overlap with the first range. Other examples of conflicts will be apparent. Alternatively or additionally, in some examples, the engine 314 is configured to combine the rule sets 352 and 316 into a single code base and test the combination by processing a set of test data using the combined code base. In these examples, the engine 314 is configured to deem the merge to be successful if no errors are detected and store the combined code base as the rule set 252 in the data store 250 If the engine 314 determines that the merge or test was unsuccessful, the engine 314 generates a message 430 identifying the error and communicates the message 430 to the interface 304. If the engine 310 determines that the merge and test were successful, the engine 314 stores the rule set 252 in the data store 250.

Continuing with the process 460, if the engine 314 communicates the message 430 to the interface 304, the interface 304 receives and processes the message 430. This message processing may include parsing the message 430 to identify the error, generating a message 432 identifying the error, and communicating the message 432 to the application 318 for subsequent error handling.

Continuing with the process 460, the interface 308 retrieves the rules set 252 from the data store 250 and communicates the rules set 252 to the layer 248. For instance, in one example, the interface 308 responds to a periodic HTTP post from the layer 248 with a JSON file specifying the rule set 252. Other communication methods will be apparent in view of this disclosure.

Continuing with the process 460, the layer 248 processes the rules set 252. For instance, in one example, the layer 248 parses a JSON file specifying the rule set 252 and stores the rules set 252 locally for subsequent validation processing as described herein.

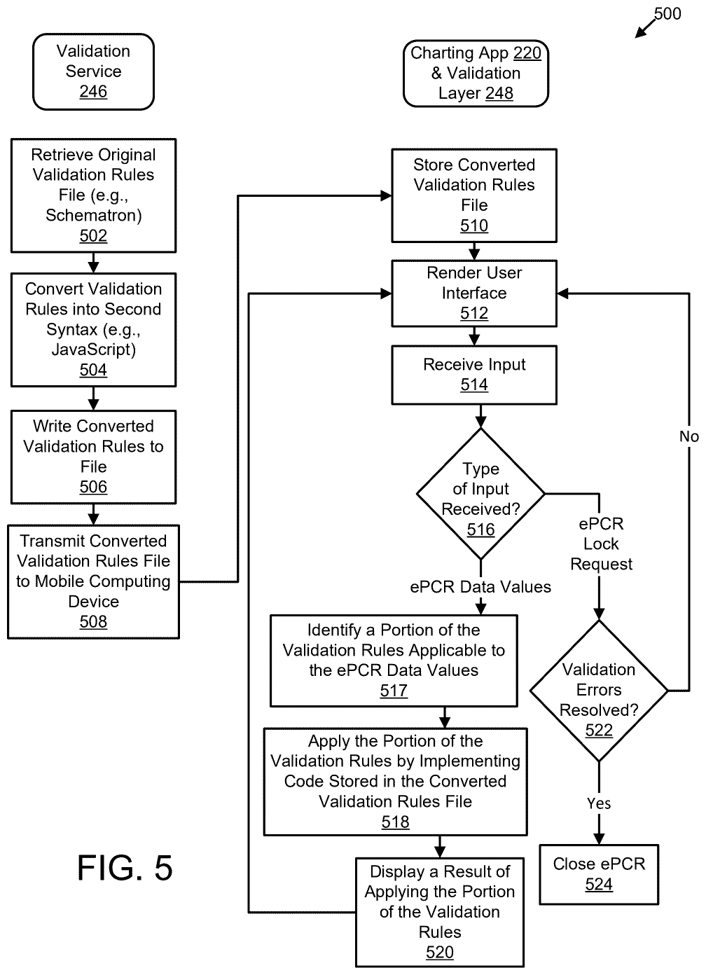

Turning now to FIG. 5, a process 500 of generating, distributing, and executing validation rule sets is illustrated. In some examples, the process 500 may be executed by an EMS charting system (e.g., the EMS charting system 200 introduced in FIGS. 2A and 2B). More specifically, portions of the process 500 may be executed by the validation service 246, the charting application 220, and the validation layer 248 introduced in FIG. 2A.

As shown in FIG. 5, the process 500 starts with the service 246 receiving 502 one or more original validation rules files. Individual validation rules files may specify one or more rule sets (e.g., the rule sets 302 and/or 316) introduced above with reference to FIG. 3B. For instance, in some examples, the service 246 downloads an electronic file specifying the rule set 302 from a URL maintained by a standards body and/or receives an electronic file specifying the rule set 316 from an administrative application (e.g., the administrative application 318 introduced in FIG. 3A).

Continuing with the process 500, the service 246 parses the one or more validation rules files to extract the one or more rule sets and converts 504 the one or more rules sets from a first syntax to a second syntax. For instance, in some examples, the service 246 transpiles the at least one rule set from the first syntax to the second syntax. In some examples, the first syntax is a Schematron syntax and the second syntax is a JAVASCRIPT syntax, although other syntaxes are possible. As part of the operation 504, the service 246 may merge the one or more rule sets into a single current rule set (e.g., the rule set 252 introduced in FIG. 2A) and may test the conversion and merge operations as described above.

Continuing with the process 500, the service 246 writes 506 the current rule set (e.g., the rule set 252 introduced in FIG. 2A) to an electronic validation file for subsequent processing. For instance, in some examples, the service 246 stores the current rule set to JSON file.

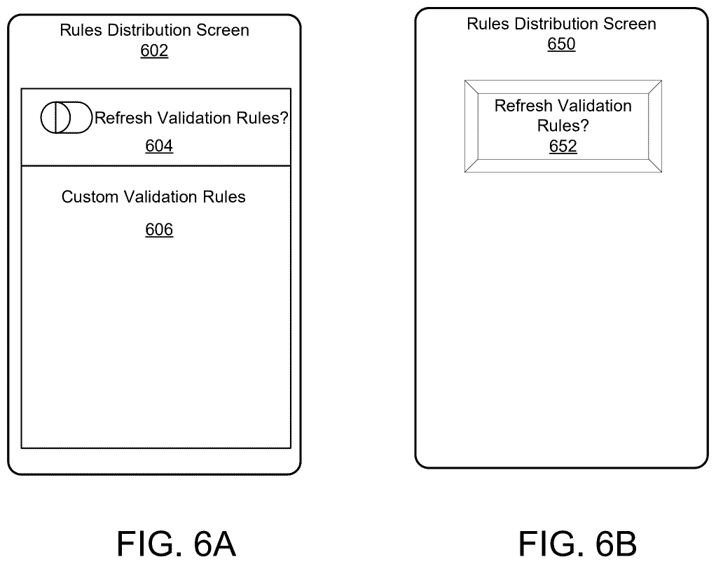

Continuing with the process 500, the service 246 communicates 508 the electronic validation file to the layer 248. In some implementations, the service 246 communicates the electronic validation file automatically and autonomously in response to a change in the electronic validation file. Alternatively or additionally, the service 246 may communicate the electronic validation file in response to a request for a specific version (e.g., a current version) of the electronic validation file. For instance, in one example, the interface 308 responds to an HTTP post from the layer 248 triggered by a user interface screen (e.g., screen 650 described below with reference to FIG. 6B) with the JSON file created in the operation 506. Alternatively or additionally, the interface 308 may respond to an inter-process communication received from the administrative interface 304 triggered by another user interface screen (e.g., screen 602 described below with reference to FIG. 6A) with the JSON file. Other communication methods will be apparent in view of this disclosure.

Continuing with the process 500, the layer 248 receives the electronic validation file and stores 510 a copy of the electronic validation file locally for subsequent processing. Further, in some examples, the layer 248 parses the electronic validation file to extract the current rule set, enumerates functions included in the current rule set, and bootstraps (e.g., links, registers, etc.) the functions to make them callable by the code that implements a charting application (e.g., the charting application 220 introduced in FIG. 2A). For instance, in some examples, the layer 248 opens and parses a JSON file to extract a textual representation of each rule in the rule set, and converts the textual representation to a callable function via, for example, a call to the Function ( ) JAVASCRIPT function. Other examples will be apparent.

Continuing with the process 500, the application 220 controls its host device (e.g., the mobile computing device 212 introduced in FIG. 2A) to render 512 an ePCR user interface. As described above, the ePCR user interface may include a variety of screens that enable the application 220 to receive user input specifying various ePCR data field values and/or requests to take other programmatic actions. These requests may include requests to navigate to a particular data set section of an ePCR and/or requests to lock the ePCR. For instance, in some examples, the application 220 controls the device 212 to render the screen 114 introduced in FIG. 1A.

Continuing with the process 500, the application 220 receives 514 input. For instance, in some examples, a EMS provider interacts with the application 220 via the ePCR user interface to authenticate and open a new ePCR to document an EMS service call.

Continuing with the process 500, as the EMS call progresses and the EMS provider continues to interact with the application 220 via the ePCR user interface, the application 220 determines types of the input received. If the application 220 determines that input specifying a data value of an ePCR data field is received (e.g., via a user interface control associated with the ePCR data value), the application 220 proceeds to operation 517. If the application 220 determines that input specifying completion of data entry and a request to lock the ePCR is received, the application 220 proceeds to operation 522. In either case, as part of the operation 516, the application 220 passes the input and control of the execution environment to the layer 248.

Continuing with the process 500, the layer 248 retrieves the electronic validation file specifying the rule set 252 and identifies 517 at least a portion thereof to apply to the input. The layer 248 may identify the portion of the rule set 252 to retrieve based on associations between one or more ePCR data fields to be validated and one or more rules within the rule set 252. The portion of the rule set 252 may include, for example, a single rule or a plurality of rules that are associated with a single ePCR data field or a plurality of ePCR data fields. Moreover, in certain implementations, the portion of the rule 252 set may include a single function corresponding to a single rule or or a plurality of functions corresponding to a plurality of rules. In addition, in some examples, the layer 248 may identify the portion of the rule set 252 to retrieve based on associations between one or more ePCR data values. For instance, in at least one example, ePCR data values that specify a pick-up and/or drop-off location of a patient may be referenced by the layer 248 to identify one or more rules to include in the portion to be applied to the input.

Continuing with the process 500, the layer 248 retrieves the electronic validation file specifying the rule set 252, or at least a portion thereof, from local storage and applies 518 the portion of the rule set 252 to the input to generate validation results. For instance, in one example, the layer 248 applies the portion of the rule set 252 by executing, via the execution environment that supports the application 220, the portion of the rule set 252. This is possible because, in these examples, the portion of the rule set 252 is encoded within the same syntax as the application 220. For instance, where the application 220 and the layer 248 are encoded as JAVASCRIPT, the portion of the rule set 252 may also be encoded as JAVASCRIPT. Moreover, in some examples, the portion of the rule set 252 is executed immediately after receipt of the input to be validated (e.g., before a focused or immediate task of the ePCR application shifts away from a control associated with an ePCR field targeted for input of a data value to another control or function of the ePCR application).

Continuing with the process 500, the layer 248 controls its host device to display 520, within the user interface, indications of the validation results generated by the operation 518. For instance, if execution of the portion of the rule set 252 yield invalid results, the portion of the rule set 252 may change features within the user interface, as described further below with reference to FIGS. 7A-8B. Next, the layer 248 passes control of the execution environment to the application 220 and the process 500 returns to the operation 512.

Continuing with the process 500, the layer 248 determines 522 whether all validation errors have been resolved. Validation errors may be triggered, for example, where a required ePCR data field has no corresponding data value or where an ePCR data field has a corresponding value that is prohibited by the applicable rules set. If validation errors remain, data entry is not complete and, therefore, must continue and the layer 248 controls the host device to render a validation control group, such as the validation control group 706 described below with reference to FIG. 7B and passes control of the execution environment to the application 220 which returns to the operation 512. If no validation errors remain, data entry is complete and the layer 248 passes control of the execution environment to the application 220, which proceeds to operation 524. In some examples, the operation 522 includes validating or revalidating some or all of the ePCR data fields against some or all of the applicable rules. It should be noted that the applicable rules may include rules from a national rule set, one or more state rule sets, and an agency rule set-among other rule sets. For instance, in at least one example, rules from the one or more state rule sets may include a first rule set associated with a first state in which a patient was picked up and a second rule set associated with a second state in which the patient was dropped off. It should be noted that such situations may involve jurisdictions other than states (e.g., counties, nations, etc.).

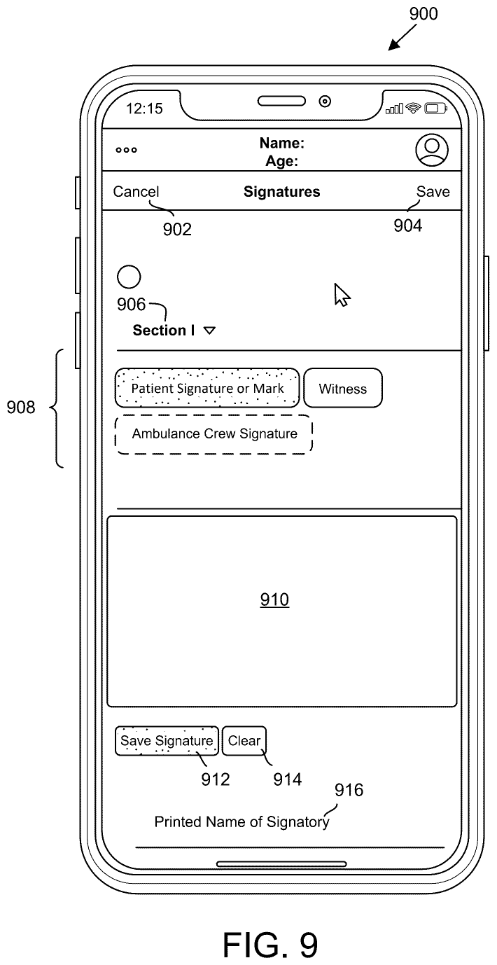

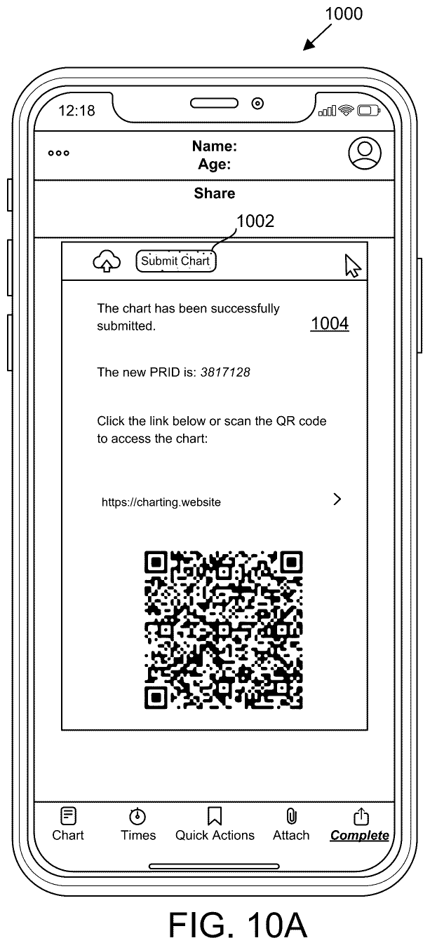

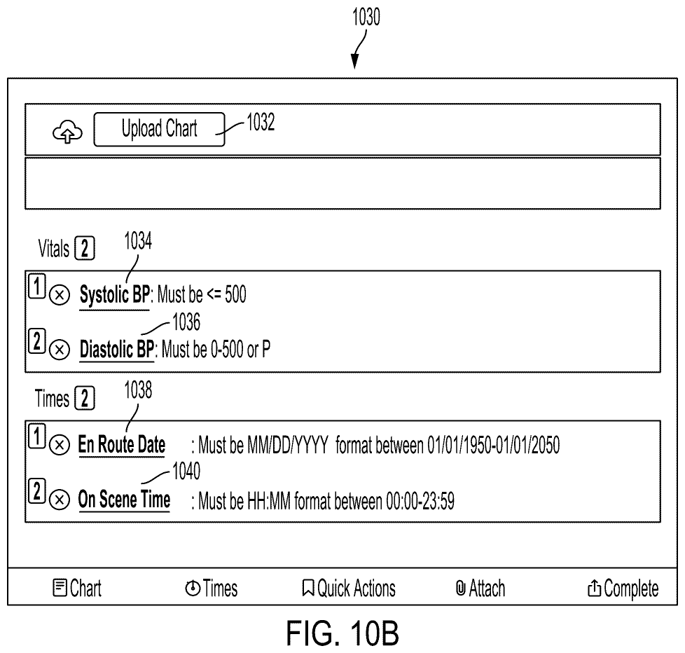

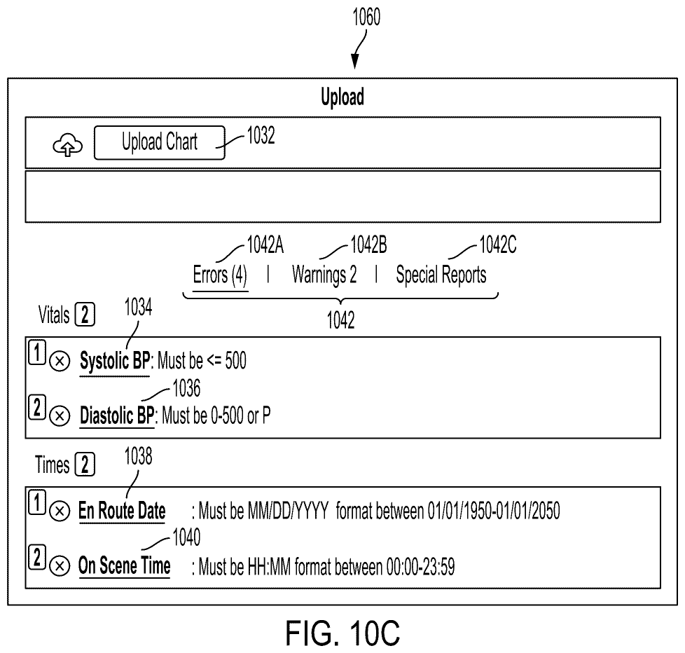

Continuing with the process 500, the application 220 finalizes and closes 524 (e.g., notifies an operating system that further access of the ePCR will not be required) the ePCR to further input by the user. In some examples, the application 220 renders the screens illustrated in FIGS. 9 and 10A-C and executes the processing described in association therewith as part of the operation 524. It should be noted that, in some examples, validation warnings may remain with regarding to the ePCR, but the warnings will not prevent the application 220 from closing the ePCR. It should also be noted that, the operations 512-524 can be executed by the device regardless of whether the device is operating in an online or offline mode (e.g., regardless of whether a network interface of the device is in a connected or disconnected state) because the computing resources needed to execute the operations 512-524 are local to the device.

User interface screens rendered by some examples will now be described with reference to FIGS. 6A-10C. It should be appreciated that these screens may be rendered by a variety of devices, such as smartphones, tablets, and other computing devices. Some examples of computing devices configured to render the screens illustrated in FIGS. 6A-10 are illustrated in FIGS. 12 and 13 and described further below. Turning now to FIG. 6A, a rules distribution screen 602 rendered by a user interface device under control of an administrative application (e.g., the administrative application 318 introduced in FIG. 3A) is illustrated. As shown in FIG. 6A, the screen 602 includes a refresh validation rules control 604 and a custom validation rules control 606.

In some examples, the application 318 is configured to receive input specifying one or more custom validation rules via the control 606 and to generate a set of custom rules based thereon. In some examples, the application 318 is configured to receive input specifying rules in human language logical implications and to generate rules based thereon. For instance, the application 318 may receive a textual sentence, such as, “If the chief complaint of a call is chest pain, then interventions must include aspirin.” In processing this logical implication, the application 318 may create validation rules, such as making a data value of “aspirin” be present in an ePCR data field listing medications given. Many other examples will be apparent in view of this disclosure. In certain examples, the application 318 is configured to store the custom rule set created from the received input in an electronic file.

In some examples, the application 318 is configured to receive, via the control 604, input requesting that a current version of the rules set administered by the validation service in communication with the application 318 be distributed to devices in the field. The application 318 is configured to generate, in response to reception such input, a request to initiate rule generation, to generate a message specifying the request, and to communicate the message to an administrative interface (e.g., the administrative interface 304 introduced in FIG. 3A) accessible by the administrative application.

Turning now to FIG. 6B, a rules distribution screen 650 rendered by a user interface device under control of a validation layer (e.g., the validation layer 248 introduced in FIG. 2A) is illustrated. As shown in FIG. 6B, the screen 650 includes a refresh validation rules button 652.

In some examples, the layer 248 is configured to receive input specifying a request to update the locally stored copy of the validation rules (e.g., an electronic validation file) via the button 652. In these examples, the layer 248 is configured to respond to such input by generating a message specifying a request to update validation rules and communicating the message to a validation interface (e.g., the validation layer interface 308 introduced in FIG. 3A). Further, in these examples, the layer 248 is configured to receive and store the validation file returned as a result of the request message.

Turning now to FIG. 7A, an initial assessment screen 700 rendered by a user interface device under control of a charting application (e.g., the charting application 220 introduced in FIG. 2A) is illustrated. As shown in FIG. 7A, the screen 700 includes a variety of controls configured to receive input specifying data values of ePCR data fields. These controls include a level of consciousness (LOC) control 704. The LOC control 704 is associated with a LOC ePCR data field, which is required to be populated by the validation rules in the instant example.

In some examples, the control 704 is selectable by the user to designate an LOC of a patient, as observed by the user. In these examples, the charting application is configured to display, in response to selection of the control 704, an enumerated list of acceptable LOC designations and to store, as a data value in an ePCR data field, an identifier of an LOC designation subsequently selected by the user from the enumerated list. For instance, the charting application may store the identifier of the selected LOC designation in a local data store (e.g., the charting data store 223 introduced in FIG. 2A).

In some examples, the charting application is configured to validate the selected LOC designation prior to shifting focus from the control 704 to another user interface control. In these examples, the charting application passes control to a validation layer (e.g., the validation layer 248 introduced in FIG. 2A) to initiate the validation. In some examples, the validation layer is an integral part of the charting application in that the validation layer includes validation code that is executable by the execution environment of the charting application. In the example illustrated in FIG. 7A, the user failed to select an LOC designation after initially selecting the control 704. As such, the validation layer determines that the data value (e.g., null) of the LOC data field is invalid because the validation code executed by the execution environment requires the LOC data field to have one of the enumerated data values. In response to determining that the data value of the LOC data field is invalid, the validation code highlights the control 704 by shading the control 704 red, thus providing an indication of validation results in proximity to (in this example, as a part of) the control 704. In certain examples, the indication of the validation results may be in proximity to the control 704 where the indication is closer to the control 704 than other controls. In some examples, the validation code further displays the reason that the data value is invalid by tagging the control 704 with the text “Required”. The reason, in some examples, may include or indicate instructions for correcting an invalid data value (e.g., include a corrective prompt). In addition, as shown in FIG. 7A, the validation code instructs the user interface to render an error control 702 that indicates a number (here, four) of unresolved validation issues present within the open ePCR or a data set section thereof. After completing these actions, the validation layer passes control back to the charting application for subsequent processing.

In some examples, the control 702 is selectable by the user to access a panel that includes controls associated with ePCR data fields for which a validation issue exists. In these examples, the charting application is configured to pass input selecting the control 702 to the validation layer for subsequent processing. The validation layer controls its host device to render a validation control group 706 as illustrated in FIG. 7B. As shown in FIG. 7B, the control group 706 includes a vitals control 708, a neurological control 710, a vitals control 712, and an assessment control 714.

Each of the controls 708-714 is associated with an ePCR data field that does not include a valid value. The validation code is configured to highlight each of the controls 708-714 by shading the control red. The validation code is further configured to display the reason that the data value stored in the control is invalid via a tag with the text descriptive of the reason and/or instructions for fixing the validation error. For instance, the control 712 specifies that “Diastolic must be within range of XX-XXX,” thereby providing a user with instructions for fixing the validation error. In some examples, each of the controls 708-714 includes an eye icon that is selectable by the user to request the charting application to display the associated control within its normal user interface screen and data set section of the ePCR. In these examples, the validation layer is configured to receive a input selecting the eye icon and, in response thereto, pass the input and control to the charting application for subsequent processing. The charting application, in turn, renders the screen through which the control is normally accessed.

FIGS. 8A and 8B illustrate examples of a user interface screen 800 in which the features described above with reference to FIGS. 7A and 7B are adapted to a smartphone or similarly space-limited device. It should be noted that, as shown in FIG. 8B, in some examples the panel of FIG. 7B that includes the controls 708-714 is replaced by a screen that utilizes the entirety of the smartphone display.

Turning now to FIG. 9, one example of a signatures screen 900 rendered by a user interface device under control of a charting application (e.g., the charting application 220 introduced in FIG. 2A) is illustrated. The screen 900 is configured to capture signatures of individuals involved in the EMS call documented by the open ePCR. Signature capture is a part of the processing required to lock the open ePCR. As shown in FIG. 9, the screen 900 includes a cancel control 902, a save control 904, a responsible section control 906, a role control group 908, a signature control 910, a save signature control 912, a clear control 914, and a printed name control 916.

In some examples, the cancel control 902 is selectable by the user to abort signature capture and navigate to the previous screen. As such, in these examples, the application 220 is configured to close, in response to selection of the control 904, the screen 900 and render the previous screen.

In some examples, the save control 904 is selectable by the user to complete signature capture and store the captured signature as ePCR data in a local data store. As such, in these examples, the application 220 is configured to store, in response to selection of the control 904, signature information previously acquired via the current instance of the screen 900 as ePCR data in the local data store.

In some examples, the responsible section control 906 is selectable by the user to designate a data set section of the ePCR for which the signatory is responsible. As such, in these examples, the application 220 is configured to interact, in response to selection of the control 906, with the user to receive an identifier of the ePCR data set section to which a signature pertains. This interaction may include display of a dropdown list, reception of input selecting an entry within the list, recordation of the selection, and display of the control 906 with the selected entry displayed therein.

In some examples, the role control group 908 is selectable by the user to designate a capacity in which the signatory is acting. As such, in these examples, the application 220 is configured to interact, in response to selection of the group 908, with the user to receive an identifier of the official capacity under which the signature is given. This interaction may include reception of input selecting a button of the group 908, recordation of the selection, and display of the group 908 with the selected button highlighted (e.g., shaded) therein. It should be noted that, in some examples, individual controls within the group 908 are optional. For instance, in the example illustrated in FIG. 9, the ambulance crew control within the group 908 is optional, as indicated by its rendering in dashed lines. Other controls may be included or excluded from the group 908 in certain examples.

In some examples, the signature control 910 is selectable by the user to record a signature. As such, in these examples, the application 220 is configured to interact, in response to selection of the control 910, with the user to receive input indicating the user's signature. This interaction may include reception of input from a stylus, finger, gesture, etc.; recordation of an image generated by tracing of the input; and display of the control 910 with the image displayed therein.

In some examples, the save signature control 912 is selectable by the user to store an image of the signature as ePCR data in a local data store. As such, in these examples, the application 220 is configured to save, in response to selection of the control 912, the recorded signature as ePCR data in the local data store.

In some examples, the clear control 914 is selectable by the user to erase a previous, unsatisfactory signature. As such, in these examples, the application 220 is configured to clear, in response to selection of the control 914, any signature presently displayed within the signature control 910.

In some examples, the printed name control 916 is selectable by the user to input the name of the signatory in text. As such, in these examples, the application 220 is configured to interact, in response to selection of the control 916, with the user to receive the signatory's name in text. This interaction may include reception of input specifying the user's signature from a keyboard, recordation of the signature, and display of the control 916 with text displayed therein.

Turning now to FIG. 10A, one example of a share screen 1000 rendered by a user interface device under control of a charting application (e.g., the charting application 220 introduced in FIG. 2A) is illustrated. In some examples, the screen 1000 is configured to lock the open ePCR upon its completion. As shown in FIG. 10A, the screen 1000 includes a submit control 1002 and a share control 1004.

In some examples, the submit control 1002 is selectable to initiate an upload of the ePCR to a remote server (e.g., a remote computing device such as, for example, one of the cloud servers 226 introduce in FIG. 2A). In these examples, the application 220 is configured to validate, in response to selection of the control 1002, the data fields of the open ePCR; upload the ePCR to the remote server; and, in response to a successful upload, display information identifying the ePCR in the share control 1004. The share control 1004 is configured to display status information and/or the information identifying the ePCR. As shown in FIG. 10A, this information can include a serial number, a URL, and/or a fiducial through which other instances of the application 220 may access the ePCR via the remote server. In some examples, the application 220 is configured to validate the data fields of the open ePCR by passing control to a validation layer (e.g., the validation layer 248 introduced in FIG. 2A).

Turning now to FIG. 10B, an upload screen 1030 rendered by a user interface device under control of a charting application (e.g., the charting application 220 introduced in FIG. 2A) is illustrated. In some examples, the screen 1030 is configured to lock the open ePCR upon its completion. As shown in FIG. 10B, the screen 1030 includes an upload control 1032 and access controls 1034-1040 that are associated with ePCR data fields for which a validation error exists.

In some examples, the application 220 is configured to validate the data fields of the open ePCR as part of initialization and rendering of the screen 1030 or in response to selection of the control 1032. In the example illustrated in FIG. 10B, the application 220 has received validation errors for some ePCR data fields and, in response thereto, rendered the access controls 1034-1040. Each of the access controls 1034-1040 includes an error icon, a link (e.g., rendered as underlined text) that can be used to access the ePCR data field associated with the control, and text explaining a reason that the data value of the ePCR data field is invalid. For instance, the control 1034 includes a link to a systolic blood pressure (BP) control configured to accept user input specifying a data value for a systolic BP data field in the open ePCR. The control 1034 further includes text explaining that the data value stored in the systolic BP data field must be less than or equal to 500. Similarly, the control 1036 includes a link to a diastolic blood pressure (BP) control configured to accept user input specifying a data value for a diastolic BP data field in the open ePCR. The control 1036 further includes text explaining that the data value stored in the diastolic BP data field must be between 0 and 500 or “P” (indicating that the diastolic pressure is 40 mm Hg less than the systolic pressure). As another example, the control 1038 includes a link to an en route date control configured to accept user input specifying data value for an en route date data field in the open ePCR. The control 1038 further includes text explaining that the data value stored in the en route date data field must be in a MM/DD/YYYY format and must be between Jan. 1, 1950 and Jan. 1, 2050. The control 1040 includes a link to an on scene time control configured to accept user input specifying a data value for an on scene time data field in the open ePCR. The control 1040 further includes text explaining that the data value stored in the on scene time data field must be in an HH: MM format and must be between 00:00 and 23:59.

In certain examples, the upload control 1032 is selectable to initiate an upload of the ePCR to a remote server (e.g., a remote computing device such as, for example, one of the cloud servers 226 introduce in FIG. 2A). Moreover, in some examples, the application 220 is configured to alter the appearance and/or accessibility of the upload control 1032 depending on whether the open ePCR has any unresolved validation issues. For instance, in some examples, the application 220 deactivates the upload control 1032 until no validation issues exist for the open ePCR. Alternatively or additionally, in some examples, the application 220 is configured to change the text displayed within the upload control 1032 from “submit” or “upload” to “complete”, “lock”, or “sign” once no validation issues exist for the open ePCR. Further, in some examples, the application 220 is configured to change the appearance and/or accessibility of the upload control 1032 once upload is initiated. For instance, in some examples, the application 220 deactivates the upload control 1032 once the upload is initiated. Alternatively or additionally, in some examples, the application 220 is configured to change the text displayed within the upload control 1032 from “submit” or “upload” to “completed”, “locked”, or “signed” once upload is initiated. It should be noted that, in some examples, upload may be initiated and completed in the foreground (e.g., while providing real time feedback regarding upload progress) or in the background (e.g., without providing real time feedback regarding upload progress).

Turning now to FIG. 10C, another example of an upload screen 1060 rendered by a user interface device under control of a charting application (e.g., the charting application 220 introduced in FIG. 2A) is illustrated. In some examples, the screen 1060 is configured to lock the open ePCR upon its completion. As shown in FIG. 10C, the screen 1060 includes the upload control 1032, the access controls 1034-1040, and a control group 1042. The control group 1402 includes validation issue type controls 1042A-1042C.

In some examples, the application 220 is configured to validate the data fields of the open ePCR as part of initialization and rendering of the screen 1060 or in response to selection of the control 1032. In the example illustrated in FIG. 10C, the application 220 has received validation errors, warnings, and special reports based on some ePCR data fields and, in response thereto, rendered the control group 1042. In various implementations, the control group 1042 may include one or more of validation errors, warning, or special reports. These validation issue type controls are examples only and other validation issue type controls corresponding to validation issues for the ePCR are within the scope of the disclosure. Further, as a configurable default option, the application 220 has rendered the access controls 1034-1040 associated with identified validation errors.

In certain examples, the application 220 is configured to receive user input selecting one of the type controls 1042A-1042C by rendering access controls for validation issues within the open ePCR of a type associated with the selected type control. As shown in the screen 1060, the error type control 1042A is selected and, as such, access controls 1034-1040 have been rendered. In certain examples, the application 220 is configured to render access controls for warnings generated by validation of the open ePCR in response to selection of the warning type control 1042B and to render access controls for special reports generated by validation of the open ePCR in response to selection of the special reports type control 1042C.

It should be noted that, in some examples, the links included in the access controls may be selectable to navigate to a screen configured to display sections of related ePCR data fields, such as a patient information section, a billing section, chief complaint section, etc. Additionally or alternatively, in some examples the access controls described above may be configured to receive and store data values for the ePCR data fields associated with the access controls without navigating to a another screen. These “in place” access controls may or may not include a link as described above. It should also be noted that, in some examples, the application 220 may be configured to operate in an override mode in which the upload control 1032 is configured to initiate an upload of the ePCR to the remote server regardless of whether unresolved validation errors exist for the open ePCR. This mode may be helpful if validation rules stored on the mobile computing device (e.g., the device 212 of FIGS. 2A and 2B) become corrupt, desynchronized with validation rules stored in the cloud environment, or otherwise contain errors.

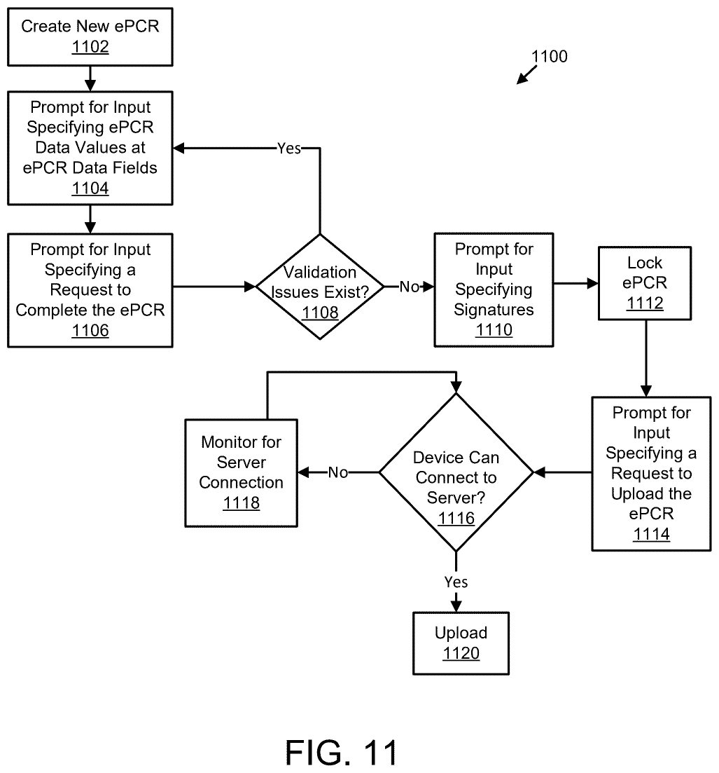

Turning now to FIG. 11, a process 1100 of creating and uploading an ePCR is illustrated. In some examples, the process 1100 may be executed by an EMS charting system (e.g., the EMS charting system 200 introduced in FIGS. 2A and 2B). More specifically, portions of the process 1100 may be executed by the validation service 246, the charting application 220, and the validation layer 248 introduced in FIG. 2A.

As shown in FIG. 11, the process 1100 starts with a charting application (e.g., the charting application 220) controlling its host device to create 1102 a new ePCR. For instance, in some examples, the charting application receives, via a user interface implemented by the host device, user input specifying a request to create a new ePCR. This user input may be, for example, a tap on a user interface control, such as the control 130 of FIG. 1A. Further, in these examples, the charting application responds to the input by allocating memory to store the new ePCR (e.g., as a JSON file). Further, in certain examples, the charting application generates an identifier of the new ePCR. This ePCR identifier may be unique (e.g. a globally unique identifier), may be generated locally, or may be generated by a cloud server process (e.g., the charting application 232 introduced in FIG. 2) and communicated to the charting application in response to an API call. Within the creation operation 1102, the charting application may store the ePCR identifier within the newly created ePCR.

Continuing with the process 1100, the charting application prompts for 1104 and receives user input specifying values of ePCR data fields. For instance, in some examples, the charting application prompts for and receives the user input via the user interface and stores the specified ePCR data field values in corresponding data fields of the ePCR. This user input may be, for example, one or more gestures interacting with screens rendered under control of the charting application, such as the screen 100 of FIG. 1B.

Continuing with the process 1100, the charting application prompts for 1106 and receives user input specifying a request to complete the ePCR. For instance, in some examples, the charting application prompts for and receives the user input via the user interface. This user input may be, for example, a tap on a user interface control, such as the “Complete” control within the navigation bar 104 introduced in FIG. 1B and further illustrated in FIGS. 7A-8A, 10A, and 10B. In response to reception of the user input, the charting application proceeds to operation 1108, in which the charting application interoperates with a validation layer (e.g., the validation layer 248) to determine 1108 whether any validation issues exist. If the charting application determines that one or more validation issues exist, the charting application returns to the operation 1104 to accept user input addressing the validation issues. If the charting application determines that no validation issues exist, the charting application proceeds to operation 1110.

Continuing with the process 1100, the charting application receives 1110 user input specifying signatures of parties involved in the patient encounter documented by the ePCR. For instance, in some examples the charting application prompts for and receives the user input via the user interface and stores the signatures in the ePCR. This user input may be, for example, one or more gestures interacting with one or more user interface controls of the charting application, such as the role control group 908, the signature control 910, and the save signature control 912 introduced in FIG. 9. In some examples, the charting application further locks 1112 the ePCR in response to saving the signatures. The charting application may execute the lock by changing a value of a dedicated status field within the ePCR to a locked value (e.g., “1”, “locked”, etc.) or the lock may be recorded as a happenstance of storing the signatures in the ePCR. Regardless of the manner in which the lock is recorded, some examples of the charting application are configured to treat a locked ePCR as immutable. In these examples, the charting application will not alter any portion of a locked ePCR. Certain examples of the charting application, however, may allow for a locked ePCR to be unlocked and revised. In this case, however, an unlocked ePCR must be resigned and relocked before subsequent processing may continue.

Continuing with the process 1100, the charting application prompts for 1114 and receives user input specifying a request to upload the ePCR. For instance, in some examples, the charting application prompts for and receives the user input via the user interface. This user input may be, for example, a tap on a user interface control, such as the submit control 1002 introduced in FIG. 10A, or the upload control 1032 introduced in FIG. 10B and further illustrated in FIG. 10C. In response to reception of the user input, the charting application proceeds to operation 1116, in which the charting application determines 1116 whether its host device is connected to a network capable of conveying the locked ePCR to a synchronization service (e.g., the service 230 introduced in FIG. 2A). This determination may involve pinging a predefined TCP/IP address, communication of an API call to establish a socket with the synchronization service, interoperating with the network interface to attempt to establish a connection, and/or or other network interface operations. If the charting application determines that such a connection exists, the charting application proceeds to operation 1120 and communicates the locked ePCR to the synchronization service via one or more API calls. These one or more API calls may, in some examples, involve conversion of an ePCR from a JSON file to one or more individual records that can be stored in a database (e.g., the charting data store 228 introduced in FIG. 2A). If the charting application determines that no such connection exists and/or an inability of the network interface to establish such a connection, the charting application proceeds to operation 1118 and monitors for a connection to be established. The operation 1118 may include, for example, shifting the host device to operate an offline mode and/or waiting for a predetermined period of time prior to returning to the operation 1116. It should be noted that, in some examples, the charting application deletes a locked ePCR from local storage upon confirmation of a successfully upload operation.

### Example Mobile Computing Device and Medical Devices

FIG. 12 illustrates one example of a mobile computing device 1200 that may be used to implement the mobile EMS charting application. In various examples, the mobile computing device 1200 is implemented using any of a variety of programmable devices (e.g., a device with data storage and at least one processor in data communication with the data storage). In some examples, the mobile computing device 1200 includes a plurality of interfaces 1204, at least one processor 1206 (e.g., at least one first processor, at least one second processor, etc.), and at least one memory device 1208 (e.g., at least one first memory, at least one second memory, etc.) coupled to one another via a communication mechanism, such as a bus. In these examples, the mobile computing device 1200 also includes a battery 1210 to power the device and may include one or more antennas 1202. The data storage device 1208 can include a non-transitory data storage medium to store an operating system and one or more software applications or programs. These programs can include instructions executable by the one or more processors to implement the features disclosed herein and the methods that they execute. The plurality of interfaces in the mobile computing device 1200 include a user interface, a network interface (e.g., at least one first network interface or at least one second network interface) configured to communicate with a network (e.g., the networks 206, 218 of FIG. 2A), and/or a medical device interface configured to exchange information with a medical device (e.g., one of the medical device(s) 214 of FIG. 2A). For instance, the plurality of interfaces 1204 may include a user interface device, such as touchscreen; a camera; an NFC tag; and/or an RFID tag. User interface devices may also include non-touchscreen displays or other hardware capable of rendering visualizations, such as UI screens and screen elements.

Particular examples of the mobile computing device 1200 include medical devices, wearable devices, smartphones, tablet computers, and laptop computers. Wearable devices that may serve as the mobile computing device 1200 include various garments with integrated technologies, watches, anklets, necklaces, belt buckles, and glasses.

In examples where the mobile computing device 1200 is implemented via a smartphone, a dedicated software application (i.e., the mobile EMS charting application) may be downloaded to the smartphone to facilitate the interactions described herein. For instance, the mobile EMS charting application may be written for an Android, IOS, Windows, or other operating system of the smartphone.

Referring to FIG. 13, a block diagram of examples of computing and medical device components are shown schematically.

The medical device 132 can include a processor 1321, a memory 221, one or more output devices 1330, one or more user input devices 244, and a communications interface 285. The communications interface 285 can include any of a variety of transmitters and/or receivers. For instance, in some examples, the communications interface 285 includes one or more of an NFC tag, an RFID tag, a barcode, and a QR code.

In various implementations, the medical devices 132 can include a defibrillator, patient monitor, defibrillator/monitor, an automated compression device, a therapeutic cooling device, an extracorporeal membrane oxygenation (ECMO) device, a ventilation device, combinations thereof, or another type of medical device configured to couple to one or more therapy delivery components to provide therapy to the patient. In an implementation, the medical devices 132 can include an integrated therapy delivery/monitoring device within a single housing 280. The single housing 280 can surround, at least in part, a patient interface device signal processor 1356 and/or a therapy delivery control module 255.

The patient interface devices 1190 can include one or more therapy delivery component(s) 261a and/or one or more sensor device(s) 261b. The medical device 132 can be configured to couple to the one or more therapy delivery component(s) 261a. In combination, the medical device 132 and the one or more therapy delivery components can provide therapeutic treatment to a patient. In an implementation, the medical device 132 can include or incorporate the therapy delivery component(s) 261a. The therapy delivery component(s) 261a are configured to deliver therapy to the patient and can be configured to couple to the patient. For example, the therapy delivery component(s) 261a can include one or more of electrotherapy electrodes including defibrillation electrodes and/or pacing electrodes, chest compression devices (e.g., one or more belts or a piston), ventilation devices (e.g., a mask and/or tubes), drug delivery devices, etc. The medical device 132 can include the one or more therapy delivery component(s) 261a and/or can be configured to couple to the one or more therapy delivery component(s) 261a in order to provide medical therapy to the patient. The therapy delivery component(s) 261a can be configured to couple to the patient. For example, a care provider may attach the electrodes to the patient, and the medical device 132 (e.g., a defibrillator or defibrillator/patient monitor) may provide electrotherapy to the patient via the defibrillation electrodes. These examples are not limiting of the disclosure as other types of medical devices, therapy delivery components, sensors, and therapy are within the scope of the disclosure.

The medical devices 132 can include, for example, a therapeutic medical device capable of delivering a medical therapy. For example, the medical therapy can be electrical therapy (e.g. defibrillation, cardiac pacing, synchronized cardioversion, diaphragmatic or phrenic nerve stimulation) and the medical devices 132 can include a defibrillator, a defibrillator/monitor and/or another medical device configured to provide electrotherapy. As another example, the medical therapy can be chest compression therapy for treatment of cardiac arrest and the medical device 132 can be a mechanical chest compression device such as a belt-based chest compression device or a piston-based chest compression device. As other examples, the medical therapy can be ventilation therapy, therapeutic cooling or other temperature management, invasive hemodynamic support therapy (e.g. Extracorporeal Membrane Oxygenation (ECMO)), etc. and the medical device 132 can be a device configured to provide a respective therapy. In an implementation, the medical device 132 can be a combination of one or more of these examples. The therapeutic medical device can include patient monitoring capabilities via one or more sensors. These types of medical therapy and devices are examples only and not limiting of the disclosure.

The medical devices 132 can include, incorporate, and/or be configured to couple to the one or more sensor(s) 261b which can be configured to couple to the patient. The sensor(s) 261b are configured to provide signals indicative of sensor data to the medical device 132. The sensor(s) 261b can be configured to couple to the patient. For example, the sensor(s) 261b can include cardiac sensing electrodes, a chest compression sensor, and/or ventilation sensors. The one or more sensors 261b can generate signals indicative of physiological parameters of the patient. For example, the physiological parameters can include one or more of at least one vital sign, an ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, physical parameters as determined via ultrasound images, parameters determined via near-infrared reflectance spectroscopy, pneumography, and/or cardiography, etc. Additionally or alternatively, the one or more sensors 261b can generate signals indicative of chest compression parameters, ventilation parameters, drug delivery parameters, fluid delivery parameters, etc.

In addition to delivering therapy to the patient, the therapy delivery component(s) 261a can include, be coupled to, and/or function as sensors and provide signals indicative of sensor data (e.g., second sensor data) to the medical device 132. For example, the defibrillation electrodes can be configured as cardiac sensing electrodes as well as electrotherapy delivery devices and can provide signals indicative of transthoracic impedance, ECG, heart rate and/or other physiological parameters. As another example, a therapeutic cooling device can be an intravenous cooling device. Such a cooling device can include an intravenous (IV) device as a therapy delivery component configured to deliver cooling therapy and sense the patient's temperature. For example, the IV device can be a catheter that includes saline balloons configured to adjust the patient's temperature via circulation of temperature controlled saline solution. In addition, the catheter can include a temperature probe configured to sense the patient's temperature. As a further example, an IV device can provide therapy via drug delivery and/or fluid management. The IV device can also monitor and/or enabling monitoring of a patient via blood sampling and/or venous pressure monitoring (e.g., central venous pressure (CVP) monitoring).

The medical devices 132 can be configured to receive the sensor signals (e.g., from the therapy delivery component(s) 261a and/or the sensor(s) 261b) and to process the sensor signals to determine and collect the patient data. The patient data can include patient data which can characterize a status and/or condition of the patient (e.g., physiological data such as ECG, heart rate, respiration rate, temperature, pulse oximetry, non-invasive hemoglobin parameters, capnography, oxygen saturation (SpO2), end tidal carbon dioxide (EtCO2), invasive blood pressure (IBP), non-invasive blood pressures (NIBP), tissue pH, tissue oxygenation, Near Infrared Spectroscopy (NIRS) measurements, etc.). Additionally or alternatively, the patient data can characterize the delivery of therapy (e.g., chest compression data such as compression depth, compression rate, etc.) and/or the patient data can characterize a status and/or condition of the medical equipment used to treat the patient (e.g., device data such as shock time, shock duration, attachment of electrodes, power-on, etc.).

In some examples, the components of 1321, 221, 1330, 244, 285, and 255 of the medical device 132 are communicatively coupled (directly and/or indirectly) to each other for bi-directional communication.

Although shown as separate entities in FIG. 13, the one or more of the components of the medical device 132 can be combined into one or more discrete components and/or can be part of the processor 1321. The processor 1321 and the memory 221 can include and/or be coupled to associated circuitry to perform the functions described herein.

In an implementation, the medical devices 132 can include a therapeutic medical device configured to deliver medical therapy to the patient. Thus, the medical devices 132 can optionally include the therapy delivery control module 255. For example, the therapy delivery control module 255 can be an electrotherapy delivery circuit that includes one or more capacitors configured to store electrical energy for a pacing pulse or a defibrillating pulse. The electrotherapy delivery circuit can further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 255 can be a compression device electro-mechanical controller configured to control a mechanical compression device. As a further example, the therapy delivery control module 255 can be an electro-mechanical controller configured to control drug delivery, temperature management, ventilation, and/or other type of therapy delivery. Alternatively, some examples of the medical devices 132 may not be configured to deliver medical therapy to the patient but can be configured to provide patient monitoring and/or diagnostic care. As shown in FIG. 13, in some examples, the therapy delivery control module 255 exchanges messages 1180 with the charting device 175 (e.g., the patient charting device). These messages can include patient data descriptive of therapy provided to the patient or other patient data stored on the medical devices 132. This patient data can be used by an ePCR application in generating an ePCR documenting a dispatched EMS event.

The medical devices 132 can incorporate and/or be configured to couple to one or more patient interface devices 1190. The patient interface devices 1190 can include one or more therapy delivery component(s) 261a and one or more sensor(s) 261b. The one or more therapy delivery component(s) 261a and the one or more sensor(s) 261b sensor can provide one or more signals to the medical devices 132 via wired and/or wireless connection(s).

The one or more therapy delivery component(s) 261a can include electrotherapy electrodes (e.g., the electrotherapy electrodes 266a), ventilation device(s) (e.g., the ventilation devices 266b), intravenous device(s) (e.g., the intravenous devices 266c), compression device(s) (e.g., the compression devices 266d), etc. For example, the electrotherapy electrodes can include defibrillation electrodes, pacing electrodes, and/or combinations thereof. The ventilation devices can include a tube, a mask, an abdominal and/or chest compressor (e.g., a belt, a cuirass, etc.), etc. and combinations thereof. The intravenous devices can include drug delivery devices, fluid delivery devices, and combinations thereof. The compression devices can include mechanical compression devices such as abdominal compressors, chest compressors, belts, pistons, and combinations thereof. In various implementations, the therapy delivery component(s) 261a can be configured to provide sensor data and/or be coupled to and/or incorporate sensors. For example, the electrotherapy electrodes can provide sensor data such as transthoracic impedance, ECG, heart rate, etc. Further the electrotherapy electrodes can include and or be coupled to a chest compression sensor. As another example, the ventilation devices can be coupled to and/or incorporate flow sensors, gas species sensors (e.g., oxygen sensor, carbon dioxide sensor, etc.), etc. As a further example, the intravenous devices can be coupled to and/or incorporate temperature sensors, flow sensors, blood pressure sensors, etc. As yet another example, the compression devices can be coupled to and/or incorporate chest compression sensors, patient position sensors, etc. The therapy delivery control module 255 can be configured to couple to and control the therapy delivery component(s) 261a.

In various implementations, the sensor(s) 261b can include one or more sensor devices configured to provide sensor data that includes, for example, but not limited to ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, images and/or videos via ultrasound, laryngoscopy, and/or other medical imaging techniques, near-infrared reflectance spectroscopy, pneumography, cardiography, and/or patient movement. Images and/or videos can be two-dimensional or three-dimensional.

The sensor(s) 261b can include sensing electrodes (e.g., the sensing electrodes 262), ventilation sensors (e.g., the ventilation sensors 264), temperature sensors (e.g., the temperature sensor 267), chest compression sensors (e.g., the chest compression sensor 268), etc. For example, the sensing electrodes can include cardiac sensing electrodes. The cardiac sensing electrodes can be conductive and/or capacitive electrodes configured to measure changes in a patient's electrophysiology, for example to measure the patient's ECG information. In an implementation, the sensing electrodes can be configured to measure the transthoracic impedance and/or a heart rate of the patient. The ventilation sensors can include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, and combinations thereof. The temperature sensors can include an infrared thermometer, a contact thermometer, a remote thermometer, a liquid crystal thermometer, a thermocouple, a thermistor, etc. and can measure patient temperature internally and/or externally. The chest compression sensor can include one or more motion sensors including, for example, one or more accelerometers, one or more force sensors, one or more magnetic sensors, one or more velocity sensors, one or more displacement sensors, etc. The chest compression sensor can be, for example, but not limited to, a compression puck, a smartphone, a hand-held device, a wearable device, etc. The chest compression sensor can be configured to detect chest motion imparted by a rescuer and/or an automated chest compression device (e.g., a belt system, a piston system, etc.). The chest compression sensor can provide signals indicative of chest compression data including displacement data, velocity data, release velocity data, acceleration data, compression rate data, dwell time data, hold time data, blood flow data, blood pressure data, etc. In an implementation, the sensing electrodes and/or the electrotherapy electrodes can include or be configured to couple to the chest compression sensor.

Continuing with FIG. 13, examples of components of the charting device 175 are shown schematically. In an implementation, the charting device 175 can be configured as a computing device. The charting device 175 can include a processor 427, a memory 421, one or more output devices 437, one or more user input devices 447, and a communications interface 445. FIG. 13 also illustrates schematic examples of components of the computing device 107. As shown in FIG. 13, the computing device 107 can include a processor 1320, a memory 321, one or more output devices 380, one or more user input devices 344, and a communications interface 345. FIG. 13 further illustrates schematic examples of components of the server(s) 1308. As shown in FIG. 13, the server(s) 1308 can include a processor 560, a memory 521, one or more output devices 530, one or more user input devices 544, and a communications interface 545.

Each of the charting device 175 (e.g., the charting device) and the computing device 107 can be a computer system, such as a desktop, notebook, mobile, portable, or other type of computing system. Each of these devices 175 and 107 can include server(s) and/or access server(s) via a monitor and/or other connected user interface device. Although described as server(s), the server(s) 1308 can be another type of computing system including for example a desktop, notebook, mobile, portable, or other type of computing system.

As shown in FIG. 13, each of the devices 175 and 107, along with the server(s) 1308 and the medical devices 132, includes a bus or other interconnection mechanism that communicably couples the processor, memory, output devices, input devices, and communication interface included therein. The bus can include a PCI/PCI-X or SCSI based system bus depending on the storage devices used, for example.

The processors 1321, 1320, 427, and 560 can each include a processor, such as, but not limited to, an Intel® Itanium® or Itanium 2® processor(s), or AMD® Opteron® or Athlon MP® processor(s), or any of a Motorola® line of processors. The communication interfaces 285, 345, 445, and 545 can each be any of an RS-232 port for use with a modem-based dialup connection, a 10/100 Ethernet port, or a Gigabit port using copper or fiber, for example. The communication interfaces 285, 345, 445, and 545 may be chosen depending on a network(s) such a Local Area Network (LAN), Wide Area Network (WAN), or any network to which the medical devices 132, the charting device 175, the computing device 107, and/or the server(s) 1308 may connect. The memories 221, 321, 421, and 521 can be Random Access Memory (RAM), Read Only Memory (ROM), Flash memory, and/or another dynamic volatile and/or non-volatile storage device(s). The memories 221, 321, 421, and 521 can be used to store information and instructions. For example, hard disks such as the Adaptec® family of SCSI drives, an optical disc, an array of disks such as RAID (e.g. the Adaptec family of RAID drives), or any other mass storage devices may be used. The components described above are meant to exemplify some types of possibilities. In no way should the aforementioned examples limit the scope of the disclosure. The memories 221, 321, 421, and 521 can further include removable storage media such as external hard-drives, floppy drives, flash drives, IOMEGA® Zip Drives, Compact Disc-Read Only Memory (CD-ROM), Compact Disc-Re-Writable (CD-RW), or Digital Video Disk-Read Only Memory (DVD-ROM), for example.

Continuing with FIG. 13, the server(s) 1308 can include, for example, the one or more storage server(s) and one or more application server(s). In some examples, the server(s) 1308 are configured to exchange messages 1170 with the computing device 107. These messages can include charting and/or case data as described above. In some examples, the server(s) 1308 are configured to exchange messages 1160 with the charting device 175 via an ePCR API. These messages can include data descriptive of ePCRs generated by the charting device 175. In some examples, the server(s) 1308 are configured to exchange messages 1195 with the medical devices 132. These messages can include data descriptive of a patient being treated via the medical device and/or treatment being delivered by the medical devices 132.

Some examples of the present disclosure include various steps, some of which can be performed by hardware components or can be embodied in machine-executable instructions. These machine-executable instructions can be stored on a non-transitory data storage medium and can be used to cause a general-purpose or a special-purpose processor programmed with the instructions to perform the steps. The non-transitory data storage medium can further store an operating system and the machine-executable instructions can be included within one or more software applications or programs, such as the ePCR application. These programs can implement the features disclosed herein and the methods that they execute. Alternatively, the steps can be performed by a combination of hardware, software, and/or firmware, on one device and/or distributed across multiple devices and/or processors. In addition, some examples of the present disclosure can be performed or implemented, at least in part (e.g., one or more modules), on one or more computer systems, mainframes (e.g., IBM mainframes such as the IBM zSeries, Unisys ClearPath Mainframes, HP Integrity NonStop server(s), NEC Express series, and others), or client-server type systems. In addition, specific hardware aspects of examples of the present disclosure can incorporate one or more of these systems, or portions thereof.

The physical processors described herein are physical processors (i.e., an integrated circuit configured to execute operations on a respective device as specified by software and/or firmware stored in a computer storage medium) operably coupled, respectively, to at least one memory device. The processors may be intelligent hardware devices (for example, but not limited to, a central processing unit (CPU), a graphics processing unit (GPU), a neural processing unit (NPU), one or more microprocessors, a controller or microcontroller, an application specific integrated circuit (ASIC), a digital signal processor (DSP), etc.) designed to perform the functions described herein and operable to carry out instructions on a respective device. Each of the processors may be one or more processors and may be implemented as a combination of hardware devices (e.g., a combination of DSP and a microprocessor, a plurality of microprocessors, one or more microprocessors in conjunction with a DSP core, or another such configuration). Each of the processors may include multiple separate physical entities that may be distributed in an associated computing device. Each of the processors is configured to execute processor-readable, processor-executable software code containing one or more instructions or code for controlling the processors to perform the functions as described herein. The processors may utilize various architectures including but not limited to a complex instruction set computer (CISC) processor, a reduced instruction set computer (RISC) processor, or a minimal instruction set computer (MISC). In various implementations, each processor may be a single-threaded or a multi-threaded processor. The processors may be, for example, but not limited to, an Intel® Itanium® or Itanium 2® processor(s), AMD® Opteron®, Athlon MP® processor(s), a Motorola® line of processor, or an ARM, Intel Pentium Mobile, Intel Core i5 Mobile, AMD A6 Series, AMD Phenom II Quad Core Mobile, or like devices.

The memories refer generally to a computer storage medium, including but not limited to RAM, ROM, FLASH, disc drives, fuse devices, and portable storage media, such as Universal Serial Bus (USB) flash drives, etc. Each of the memories may include, for example, random access memory (RAM), or another dynamic storage device(s) and may include read only memory (ROM) or another static storage device(s) such as programmable read only memory (PROM) chips for storing static information such as instructions for a coupled processor. Each memory may include USB flash drives that may store operating systems and other applications. The USB flash drives may include input/output components, such as a wireless transmitter and/or USB connector that can be inserted into a USB port of another computing device. Each memory may be long term and/or short term and is not to be limited to a particular type of memory or number of memories, or type of media upon which memory is stored. Each memory includes a non-transitory processor-readable storage medium (or media) that stores the processor-readable, processor-executable software code. Each memory may store information and instructions. For example, each memory may include flash memory and/or another storage media may be used, including removable or dedicated memory in a mobile or portable device. As another example, hard disks such as the Adaptec® family of SCSI drives, an optical disc, an array of disks such as RAID (e.g. the Adaptec family of RAID drives), or another mass storage device may be used. Each memory may include removable storage media such as, for example, external hard-drives, floppy drives, flash drives, zip drives, compact disc-read only memory (CD-ROM), compact disc-re-writable (CD-RW), or digital video disk-read only memory (DVD-ROM.

Communicatively coupled devices as described herein may transmit and/or receive information via a wired and/or wireless communicative coupling. The information may include information stored in at least one memory. The information may include, for example, but not be limited to, resuscitative treatment information, physiological information, patient information, rescuer and/or caregiver information, location information, rescue and/or medical treatment center information, etc. The communicative couplings may enable short-range and/or long-range wireless communication capabilities which may include communication via near field communication, ZIGBEE, Wi-Fi, BLUETOOTH, satellite(s), radio waves, a computer network (e.g., the Internet), a cellular network, a LAN, WAN, a mesh network, an ad hoc network, or another network. The communicative couplings may include, for example, an RS-232 port for use with a modem based dialup connection, a copper or fiber 10/100/1000 Ethernet port, or a BLUETOOTH or Wi-Fi interface.

Displays as described herein may provide a user interface (UI). A particular display may be, for example, but not be limited to, a touchscreen display, an AR display, a liquid crystal display (LCD), and/or a light emitting diode (LED) display. The touchscreen may be, for example, a pressure sensitive touchscreen or a capacitive touchscreen. The touchscreen may capture user input provided via touchscreen gestures and/or provided via exertions of pressure on a particular area of the screen. The displays may provide visual representations of data captured by and/or received at the medical device 132. The visual representations may include still images and/or video images (e.g., animated images).

The computing devices referred to herein may include one or more user input devices such as, for example, a keyboard, a mouse, joystick, trackball, or other pointing device, a microphone, a camera, etc. In an implementation, the user input devices may be configured to capture information, such as, for example, patient medical history (e.g., medical record information including age, gender, weight, body mass index, family history of heart disease, cardiac diagnosis, co-morbidity, medications, previous medical treatments, and/or other physiological information), physical examination results, patient identification, caregiver identification, healthcare facility information, etc.

The processor, memory, communication interfaces, input and/or output devices and other components described above are meant to exemplify some types of possibilities. In no way should the aforementioned examples limit the scope of the disclosure, as they are only exemplary embodiments of these components.

Various modifications and additions can be made to the exemplary embodiments discussed without departing from the scope of the present disclosure. For example, while the embodiments described above refer to particular features, the scope of the disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

It should be noted that the devices described herein can be used in medical settings other than EMS. For instance, some examples can be useful in hospital, clinic, military medical treatment, home, and other non-EMS settings. It should also be noted that EMS care can include both emergency care (e.g., car accident, cardiac arrest, overdose, etc.) and scheduled non-emergency care like a transport for dialysis, chemotherapy, physical therapy, and the like.

Having thus described several aspects of at least one example, it is to be appreciated that various alterations, modifications, and improvements will readily occur to those skilled in the art. For instance, examples disclosed herein can also be used in other contexts. Such alterations, modifications, and improvements are intended to be part of this disclosure and are intended to be within the scope of the examples discussed herein. Accordingly, the foregoing description and drawings are by way of example only.

1.-96. (canceled)

97. A system for locally validating an electronic patient care record (ePCR), the system comprising:
a server configured to
receive a standard set of rules encoded in a first syntax,
convert the standard set of rules from the first syntax to a second syntax that is executable within an execution environment, and
communicate the standard set of rules encoded in the second syntax to a mobile computing device configured to store an ePCR application encoded in the second syntax; and
the mobile computing device,
wherein the mobile computing device is configured to
operate in an online mode,
receive the standard set of rules encoded in the second syntax while operating in the online mode,
shift to operate in an offline mode,
execute, within the execution environment while operating in the offline mode, at least a portion of the ePCR application to receive at least one ePCR data value via at least one user interface control,
execute, within the execution environment while operating in the offline mode, at least a portion of the standard set of rules encoded in the second syntax to validate the at least one ePCR data value prior to a focus of the ePCR application shifting away from the at least one user interface control, and
display an indication of whether the at least one ePCR data value is valid prior to the focus of the ePCR application shifting away from the at least one user interface control.

98. The system of claim 97, wherein:
the first syntax is a Schematron syntax; and
the second syntax is a JAVASCRIPT syntax.

99. The system of claim 98, wherein the execution environment comprises a browser or a browser container.

100. The system of claim 97, wherein the indication comprises a corrective prompt.

101. The system of claim 97, wherein the ePCR application is configured to validate and lock the ePCR regardless of a state of connection of the mobile computing device.

102. The system of claim 97, wherein:
the mobile computing device comprises
at least one memory;
at least one network interface;
at least one user interface device; and
at least one processor coupled with the at least one memory, the at least one network interface, and the at least one user interface device, the at least one processor configured to
implement the ePCR application to cause the mobile computing device to provide, at the at least one user interface device, an ePCR user interface comprising the at least one user interface control, the ePCR user interface being configured to capture data values for the ePCR, the ePCR comprising a plurality of data fields,
retrieve an electronic validation file from the at least one memory,
receive a plurality of data values comprising the at least one ePCR data value via the ePCR user interface, each data value corresponding to a respective data field of the plurality of data fields,
in response to receiving each data value, validate the data value at the mobile computing device, wherein to validate comprises to apply at least a portion of a set of rules to an input to each data field as the data values are received and prior to completion of data entry and locking of the ePCR,
generate a plurality of validation results based on validations of the data values, and
provide indications of the validation results at the ePCR user interface, the indications comprising the indication of whether the at least one ePCR data value is valid.

103. The system of claim 102, wherein the at least one processor is configured to locally validate the data value at the mobile computing device.

104. The system of claim 103, wherein the at least one processor is configured to:
locally validate the data value at the mobile computing device while the at least one network interface is in a state of disconnection from a network configured to communicatively couple the mobile computing device to a remote server.

105. (canceled)

106. The system of claim 104, wherein the at least one processor is configured to:
receive input instructing the at least one processor to lock the ePCR; and
lock the ePCR in response to reception of the input while the at least one network interface is in the state of disconnection.

107. The system of claim 102, wherein:
the electronic validation file comprises a set of rules coded in a second syntax according to which the ePCR application is coded based on a conversion from a set of rules coded according to a first syntax, wherein the first syntax is incompatible with local validation; and
wherein the local validation comprises a validation performed while the at least one network interface is in a state of disconnection from a network configured to communicatively couple the mobile computing device to a remote server.

108. The system of claim 107, wherein the first syntax is a Schematron syntax and the second syntax is a JAVASCRIPT syntax.

109.-111. (canceled)

112. The system of claim 102, wherein the at least one processor is configured to validate a data value provided in response to the indications of validation errors prior to completion of data entry and locking of the ePCR.

113. (canceled)

114. (canceled)

115. The system of claim 102, wherein the at least one processor is configured to iteratively update the indications of validation results in response to receiving each data value.

116. The system of claim 115, wherein the indications of the validation results comprise instructions for fixing a validation error.

117. The system of claim 115, wherein the indications of the validation results comprise an error control configured to:
indicate a number of the validation results associated with a particular data set section of the ePCR, and
in response to a user selection of the error control, indicate the validation results associated with the particular data set section of the ePCR.

118. The system of claim 115, wherein the at least one processor is configured to provide the indications of the validation results at the ePCR user interface in proximity to controls of the ePCR user interface associated with the validation results.

119. (canceled)

120. The system of claim 115, wherein the indications of the validation results comprise an access control configured to:
indicate a validation error associated with a data field of the plurality of data fields; and
navigate, in response to a user selection of the access control, the ePCR user interface to a data set section associated with the data field.

121. The system of claim 115, wherein the indications of the validation results comprise an access control configured to:
indicate a validation error associated with a data field of the plurality of data fields; and
receive, in response to a user selection of the access control, a new data value to store in the data field.

122. (canceled)

123. (canceled)

124. The system of claim 102, wherein the set of rules comprises standard validation rules.

125. The system of claim 124, wherein the standard validation rules are applicable to determine whether an ePCR complies with a National Emergency Medical Services Information System (NEMSIS) standard.

126. The system of claim 102, wherein the set of rules specify at least one message to be displayed if at least one validation result indicates at least one invalid value within at least one data field.

127. The system of claim 126, wherein the at least one message comprises at least one custom message specified by a healthcare provider.

128. The system of claim 102, wherein the at least one processor is configured to:
store the validation results in the memory;
detect an inability to establish a connection to a remote server via the at least one network interface;
wait for a timeout period;
establish, after the timeout period, a connection to the remote server via the at least one network interface; and
upload the validation results to the remote server.

129.-136. (canceled)

137. The system of claim 102, wherein to apply the at least the portion of the set of rules comprises to implement code stored in the electronic validation file.

138.-186. (canceled)
